SEQUENCE LISTING (35 sequences)

SEQ ID NO: 1 - protein, 125 aa, Artificial Sequence
  Synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 2 - protein, 107 aa, Artificial Sequence
  Synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 3 - DNA, 1413 bp, Artificial Sequence
  Synthetic construct
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatccgaggggagctaccctttactactactactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - DNA, 714 bp, Artificial Sequence
  Synthetic construct
atggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattaacagctatttagattggtatcagcagaaaccagggaaagcccctaaactcctgatctatgctgcatccagtttgcaaagtggggtcccatcaaggttcagtggcagtggatctgggacagatttcactctcaccatcagcagtctgcaacctgaagattttgcaacttactactgtcaacagtattacagtactccattcactttcggccctgggaccaaagtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 5 - protein, 118 aa, Artificial Sequence
  Synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 6 - protein, 108 aa, Artificial Sequence
  Synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 7 - DNA, 355 bp, Artificial Sequence
  Synthetic construct
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcag

SEQ ID NO: 8 - DNA, 325 bp, Artificial Sequence
  Synthetic construct
gaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaac

SEQ ID NO: 9 - protein, 113 aa, Artificial Sequence [sequence omitted]

SEQ ID NO: 10 - protein, 107 aa, Artificial Sequence [sequence omitted]

SEQ ID NO: 11 - DNA, 339 bp, Artificial Sequence
  Synthetic construct
caggtgcagctggtggagtctgggggaggcgtggtccagcctgggaggtccctgagactcgactgtaaagcgtctggaatcaccttcagtaactctggcatgcactgggtccgccaggctccaggcaaggggctggagtgggtggcagttatttggtatgatggaagtaaaagatactatgcagactccgtgaagggccgattcaccatctccagagacaattccaagaacacgctgtttctgcaaatgaacagcctgagagccgaggacacggctgtgtattactgtgcgacaaacgacgactactggggccagggaaccctggtcaccgtctcctca

SEQ ID NO: 12 - DNA, 321 bp, Artificial Sequence
  Synthetic construct
gaaattgtgttgacacagtctccagccaccctgtctttgtctccaggggaaagagccaccctctcctgcagggccagtcagagtgttagtagttacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagagtagcaactggcctcggacgttcggccaagggaccaaggtggaaatcaaa

SEQ ID NO: 13 - protein, 5 aa, Artificial Sequence
  Synthetic construct
SGGGG

SEQ ID NO: 14 - protein, 15 aa, Artificial Sequence
  Synthetic construct
SGGGGSGGGGSGGGG

SEQ ID NO: 15 - protein, 5 aa, Artificial Sequence
  Synthetic construct
SGGGG

SEQ ID NO: 16 - protein, 451 aa, Artificial Sequence
  Synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 214 aa, Artificial Sequence
  Synthetic construct
EIVLTQSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 12 aa, Artificial Sequence
  Synthetic construct
RASQSVGSSYLA

SEQ ID NO: 19 - protein, 7 aa, Artificial Sequence
  Synthetic construct
GAFSRAT

SEQ ID NO: 20 - protein, 9 aa, Artificial Sequence
  Synthetic construct
QQYGSSPWT

SEQ ID NO: 21 - protein, 5 aa, Artificial Sequence
  Synthetic construct
SYTMH

SEQ ID NO: 22 - protein, 18 aa, Artificial Sequence
  Synthetic construct
FISYDGSNNKYYADSVKG

SEQ ID NO: 23 - protein, 9 aa, Artificial Sequence
  Synthetic construct
TGWLGPFDY

SEQ ID NO: 24 - protein, 11 aa, Artificial Sequence
  Synthetic construct
RASQSVSSYLA

SEQ ID NO: 25 - protein, 7 aa, Artificial Sequence
  Synthetic construct
DASNRAT

SEQ ID NO: 26 - protein, 9 aa, Artificial Sequence
  Synthetic construct
QQSSNWPRT

SEQ ID NO: 27 - protein, 5 aa, Artificial Sequence
  Synthetic construct
NSGMH

SEQ ID NO: 28 - protein, 17 aa, Artificial Sequence
  Synthetic construct
VIWYDGSKRYYADSVKG

SEQ ID NO: 29 - protein, 5 aa, Artificial Sequence
  Synthetic construct
NDDYW

SEQ ID NO: 30 - protein, 211 aa, Artificial Sequence
  Synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 31 - protein, 213 aa, Artificial Sequence
  Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 4790 bp, Artificial Sequence
  Synthetic construct
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcctcgaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 33 - protein, 15 aa, Artificial Sequence
  Synthetic construct
SGGGGSGGGGSGGGG

SEQ ID NO: 34 - protein, 15 aa, Artificial Sequence
  Synthetic construct
SGGGGSGGGGSGGGG

SEQ ID NO: 35 - protein, 6 aa, Artificial Sequence
  Synthetic construct
HHHHHH

(19) United States
(12) Patent Application Publication
(10) Pub. No.: US 2016/0145355 A1
(43) Pub. Date: May 26, 2016

(54) BISPECIFIC ANTIBODIES

(71) Applicant: BIOMED VALLEY DISCOVERIES, INC., Kansas City, MO (US)

(72) Inventors: Saurabh SAHA, Wellesley Hills, MA (US); Jeffrey James ROIX, Boston, MA (US); Dean WELSCH, Parkville, MO (US)

(21) Appl. No.: 14/900,757

(22) PCT Filed: Jun. 20, 2014
(86) PCT No.: PCT/US2014/043480

§ 371 Date: Dec. 22, 2015

Related U.S. Application Data
(60) Provisional application No. 61/838,654, filed on Jun. 24, 2013.

(51) Int. Cl. C07K 16/46; C07K 16/28; C07K 16/30

(52) U.S. Cl. CPC C07K 16/468; C07K 16/30; C07K 16/3038; C07K 16/3023; C07K 16/2818; C07K 16/2803; C07K 2317/31; C07K 2317/565; C07K 2317/76; C07K 2317/21; C07K 2317/35; C07K 2317/626; A61K 2039/507

(57) ABSTRACT

The present invention provides, inter alia, bispecific antibodies containing a first antigen binding moiety that specifically binds an epitope on human cytotoxic T-lymphocyte-associated antigen 4 (CTLA-4) and a second antigen binding moiety that specifically binds an epitope on a human programmed death 1 (PD-1) receptor. Also provided are pharmaceutical compositions containing such bispecific antibodies, as well as methods and kits for treating cancer using such bispecific antibodies and pharmaceutical compositions.

### CROSS-REFERENCE TO RELATED APPLICATIONS

The present invention claims benefit to U.S. Provisional Application No. 61/838,654 filed Jun. 24, 2013. The entire contents of the above application are incorporated by reference.

### FIELD OF INVENTION

The present invention provides, inter alia, bispecific antibodies that specifically bind to both human cytotoxic T-lymphocyte-associated antigen 4 (CTLA-4) and human programmed death 1 (PD-1) receptors. Also provided are pharmaceutical compositions containing such bispecific antibodies, and methods and kits for treating cancer using such bispecific antibodies and pharmaceutical compositions.

### INCORPORATION BY REFERENCE OF SEQUENCE LISTING

This application contains references to amino acids and/or nucleic acid sequences that have been filed concurrently herewith as sequence listing text file “0345009.txt”, file size of 13 KB, created on Jun. 17, 2013. The aforementioned sequence listing is hereby incorporated by reference in its entirety pursuant to 37 C.F.R. §1.52(e)(5).

### BACKGROUND OF THE INVENTION

Antibody-mediated blockade of CTLA-4 prevents development of tolerance, augments anti-tumor responses, and exacerbates autoimmune disease. Blocking PD-1 using an anti-PD-1 antibody has shown that PD-1 acts as a negative regulator of T-cell activation. Yervoy (Ipilimumab) is a marketed anti-CTLA-4 antibody which has demonstrated improved survival in patients with metastatic melanoma. Similarly, an anti-PD-1 antibody in clinical trials has demonstrated a significant percentage of objective responses in cancer patients treated with the antibody.

Recently, reports of concurrent therapy using separate intravenous doses of nivolumab and ipilimumab were shown, in a phase 1 clinical trial, to have rapid and deep tumor regression in a substantial portion of the patients. Concurrent therapy suffers from a number of drawbacks including, inconvenience to the patient, added pain, and added difficulty of manufacturing and characterizing multiple agents.

Thus, despite the advances in medical sciences, there is still an unmet medical need for new potent agents for the treatment of cancers. The present invention is directed to meeting this and other needs.

### SUMMARY OF THE INVENTION

The inventors expect that combined bispecific anti-PD-1 and anti-CTLA-4 antibody as a single biological entity would exhibit efficacy superior to anti-PD-1 or anti-CTLA-4 molecules administered separately or in combination for the treatment of cancer. To the best of the inventors' knowledge, no such therapeutic has been made. Accordingly, it would be beneficial to provide a single therapeutic composition having both anti-CTLA-4 and anti-PD-1 antibody activity.

Thus, one embodiment of the present invention is a bispecific antibody. This bispecific antibody comprises:

(a) a first antigen binding moiety that specifically binds an epitope on human cytotoxic T-lymphocyte-associated antigen 4 (CTLA-4), the first antigen binding moiety comprising an antibody having:

(1) a heavy chain CDR1 comprising SYTMH (SEQ ID NO:21), a heavy chain CDR2 comprising FISYDGNNKYYADSVKG (SEQ ID NO:22), and a heavy chain CDR3 comprising TGWLGPFDY (SEQ ID NO:23); and

(2) a light chain CDR1 comprising RASQSVGSSYLA (SEQ ID NO:18), a light chain CDR2 comprising GAFSRAT (SEQ ID NO:19), and a light chain CDR3 comprising QQYGSSPWT (SEQ ID NO:20); and

(b) a second antigen binding moiety that specifically binds an epitope on a human programmed death 1 (PD-1) receptor, the second antigen binding moiety comprising an antibody having:

(1) a heavy chain CDR1 comprising NSGMH (SEQ ID NO:27), a heavy chain CDR2 comprising VIWYDGSKRYYADSVKG (SEQ ID NO:28), and a heavy chain CDR3 comprising NDDYW (SEQ ID NO:29); and

(2) a light chain CDR1 comprising RASQSVSSYL (SEQ ID NO:24), a light chain CDR2 comprising DASNRAT (SEQ ID NO:25), and a light chain CDR3 comprising QQSSNWPRT (SEQ ID NO:26).

Another embodiment of the present invention is a bispecific antibody. This bispecific antibody comprises:

(a) a first antigen binding moiety that specifically binds an epitope on human cytotoxic T-lymphocyte-associated antigen 4 (CTLA-4), and

(b) a second antigen binding moiety that specifically binds an epitope on a human programmed death 1 (PD-1) receptor.

An additional embodiment of the present invention is a pharmaceutical composition. This pharmaceutical composition comprises any bispecific antibody disclosed herein and a pharmaceutically acceptable excipient.

A further embodiment of the present invention is a method of treating cancer in a subject. This method comprises administering to the subject a therapeutically effective amount of any pharmaceutical composition disclosed herein.

Another embodiment of the present invention is a method of treating cancer in a subject. This method comprises administering to the subject a therapeutically effective amount of a bispecific antibody, one antigen binding moiety of which specifically binds human CTLA-4 and the other antigen binding moiety of which binds to human PD-1 receptor.

An additional embodiment of the present invention is a method of treating melanoma in a subject. This method comprises administering to the subject a therapeutically effective amount of at least one isolated bispecific antibody comprising a first antigen binding moiety that specifically binds an epitope in the extracellular Ig V domain of the human CTLA-4 and a second antigen binding moiety that specifically binds an epitope in the extracellular Ig V domain of the human PD-1 receptor.

A further embodiment of the present invention is a kit for treating a cancer in a subject. This kit comprises any pharmaceutical composition disclosed herein.

Another embodiment of the present invention is a bispecific antibody. This bispecific antibody comprises:

(a) a first antigen binding moiety that specifically binds an epitope on human cytotoxic T-lymphocyte-associated antigen 4 (CTLA-4), and

(b) a second antigen binding moiety that specifically binds an epitope on a human programmed death ligand 1 (PD-L1).

An additional embodiment of the present invention is a method of treating cancer in a subject. This method comprises administering to the subject a therapeutically effective amount of a bispecific antibody, one antigen binding moiety of which specifically binds human CTLA-4 and the other antigen binding moiety of which binds to human PD-L1.

### BRIEF DESCRIPTION OF THE DRAWINGS

FIG. 1 shows the construction of a bispecific single chain diabody expression vector.

FIG. 2 shows the construction of segments of two expression vectors for a trivalent bispecific antibody (tribody) containing 2 scFvs that recognize CTLA-4 and one Fab that recognizes PD-1.

FIG. 3 shows a schematic of the trivalent bispecific antibody.

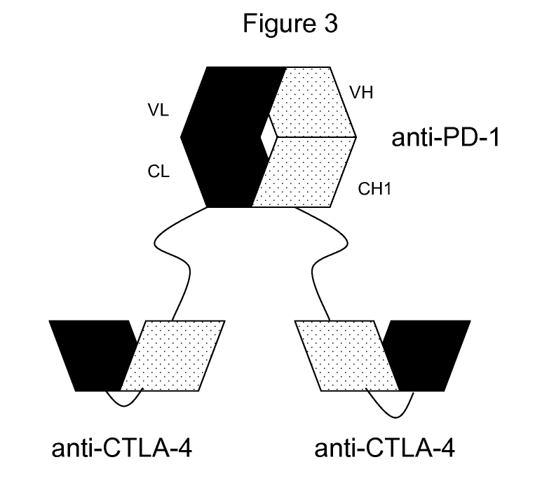

### DETAILED DESCRIPTION OF THE INVENTION

One embodiment of the present invention is a bispecific antibody. This bispecific antibody comprises:

(a) a first antigen binding moiety that specifically binds an epitope on human cytotoxic T-lymphocyte-associated antigen 4 (CTLA-4), the first antigen binding moiety comprising an antibody having:

(1) a heavy chain CDR1 comprising SYTMH (SEQ ID NO:21), a heavy chain CDR2 comprising FISYDGNNKYYADSVKG (SEQ ID NO:22), and a heavy chain CDR3 comprising TGWLGPFDY (SEQ ID NO:23); and

(2) a light chain CDR1 comprising RASQSVGSSYLA (SEQ ID NO:18), a light chain CDR2 comprising GAFSRAT (SEQ ID NO:19), and a light chain CDR3 comprising QQYGSSPWT (SEQ ID NO:20); and

(b) a second antigen binding moiety that specifically binds an epitope on a human programmed death 1 (PD-1) receptor, the second antigen binding moiety comprising an antibody having:

(1) a heavy chain CDR1 comprising NSGMH (SEQ ID NO:27), a heavy chain CDR2 comprising VIWYDGSKRYYADSVKG (SEQ ID NO:28), and a heavy chain CDR3 comprising NDDYW (SEQ ID NO:29); and

(2) a light chain CDR1 comprising RASQSVSSYL (SEQ ID NO:24), a light chain CDR2 comprising DASNRAT (SEQ ID NO:25), and a light chain CDR3 comprising QQSSNWPRT (SEQ ID NO:26).

As used herein, an “antibody” encompasses naturally occurring immunoglobulins (e.g., IgM, IgG, IgD, IgA, IgE, etc.) as well as non-naturally occurring immunoglobulins, including, for example, single chain antibodies, chimeric antibodies (e.g., humanized murine antibodies) and heteroconjugate antibodies (e.g., bispecific antibodies), as well as antigen-binding fragments thereof, (e.g., Fab′, F(ab′)₂, Fab, Fv, and rIgG). See also, e.g., Pierce Catalog and Handbook, 1994-1995 (Pierce Chemical Co., Rockford, Ill.); Kuby, J., Immunology, 3rd Ed., W.H. Freeman & Co., New York (1998). Non-naturally occurring antibodies can be constructed using solid phase peptide synthesis, can be produced recombinantly, or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies, are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995); each of which is incorporated herein by reference).

Full length antibodies can be proteolytically digested down to several discrete, functional antibody fragments, which retain the ability to recognize the antigen. For example, the enzyme papain can be used to cleave a full length immunoglobulin into two Fab fragments and an Fc fragment. Thus, the Fab fragment is typically composed of two variable domains and two constant domains from the heavy and light chains. The Fv region is usually recognized as a component of the Fab region and typically comprises two variable domains, one from each of the heavy (V_{H}) and light (V_{L}) chains. The enzyme pepsin cleaves below the hinge region, so a F(ab′)₂ fragment and a pFc′ fragment is formed. F(ab′)₂ fragments are intact antibodies that have been digested, removing the constant (Fc) region. Two Fab′ fragments can then result from further digestion of F(ab′)₂ fragments. As used herein, “antibody fragments” means the a portion of the full length antibody that retains the ability to recognize the antigen, as well as various combinations of such portions. Examples of antibody fragments include, but are not limited to, Fv, Fab, Fab′, Fab′-SH, F(ab′)₂, diabodies, tribodies, scFvs, and single-domain antibodies (dAbs). Diabodies, tribodies, scFvs, and dAbs are discussed in detail below.

Typically, a full length antibody has at least one heavy and at least one light chain. Each heavy chain contains a variable domain (V_{H}) and typically three or more constant domains (C_{H}1, C_{H}2, C_{H}3, etc.), while each light chain contains a variable domain (V_{L}) and a constant domain C_{L}. Light and heavy chain variable regions contain four “framework” regions interrupted by three hypervariable regions, also called “complementarity-determining regions” or “CDRs”. The extent of the framework regions and CDRs have been defined. See, e.g., Kabat et al., U.S. Dept. of Health and Human Servies, Sequences of Proteins of Immunological Interest (1983) and Chothia et al., J. Mol. Biol. 196:901-917 (1987). The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody, whereas a V_{L} CDR1 is the CDR1 from the variable domain of the light chain of the antibody.

The term “bispecific antibody” refers to an antibody having the capacity to bind to two distinct epitopes either on a single antigen or two different antigens. As used herein, “epitope” or “antigenic determinant” refers to a site on an antigen to which an antibody binds. Epitopes can be formed both from contiguous amino acids (linear epitope) or noncontiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). A preferred method for epitope mapping is surface plasmon resonance.

Bispecific antibodies of the present invention can be produced via biological methods, such as somatic hybridization; or genetic methods, such as the expression of a non-native DNA sequence encoding the desired antibody structure in an organism; chemical methods, such as chemical conjugation of two antibodies; or a combination thereof (Kontermann, R. E. In: Bispecific Antibodies. Kontermann R E (ed.), Springer Heidelberg Dordrecht London New York, pp. 1-28 (2011)).

Chemically conjugated bispecific antibodies arise from the chemical coupling of two existing antibodies or antibody fragments. Typical couplings include cross-linking two different full-length antibodies, cross-linking two different Fab′ fragments to produce a bispecific F(ab′)₂, and cross-linking a F(ab′)₂ fragment with a different Fab′ fragment to produce a bispecific F(ab′)₃. For chemical conjugation, oxidative reassociation strategies can be used. Current methodologies include the use of the homo- or heterobifunctional cross-linking reagents (Id.).

Heterobifunctional cross-linking reagents have reactivity toward two distinct reactive groups on, for example, antibody molecules. Examples of heterobifunctional cross-linking reagents include SPDP (N-succinimidyl-3-(2-pyridyldithio)propionate), SATA (succinimidyl acetylthioacetate), SMCC (succinimidyl trans-4-(maleimidylmethyl)cyclohexane-1-carboxylate), EDAC (1-ethyl-3-(3-dimethylaminopropyl) carbodiimide), PEAS (N-((2-pyridyldithio)ethyl)-4-azidosalicylamide), ATFB, SE (4-azido-2,3,5,6-tetrafluorobenzoic acid, succinimidyl ester), benzophenone-4-maleimide, benzophenone-4-isothiocyanate, 4-benzoylbenzoic acid, succinimidyl ester, iodoacetamide azide, iodoacetamide alkyne, Click-iT maleimide DIBO alkyne, azido (PEO)4 propionic acid, succinimidyl ester, alkyne, succinimidyl ester, Click-iT succinimidyl ester DIBO alkyne, Sulfo-SBED (Sulfo-N-hydroxysuccinimidyl-2-(6-[biotinamido]-2-(p-azido benzamido)-hexanoamido)ethyl-1,3′-dithioproprionate), photoreactive amino acids (e.g., L-Photo-Leucine and L-Photo-Methionine), NHS-haloacetyl crosslinkers such as, for example, Sulfo-SIAB, SIAB, SBAP, SIA, NHS-maleimide crosslinkers such as, for example, Sulfo-SMCC, SM(PEG)n series crosslinkers, SMCC, LC-SMCC, Sulfo-EMCS, EMCS, Sulfo-GMBS, GMBS, Sulfo-KMUS, Sulfo-MBS, MBS, Sulfo-SMPB, SMPB, AMAS, BMPS, SMPH, PEG12-SPDP, PEG4-SPDP, Sulfo-LC-SPDP, LC-SPDP, SMPT, DCC (N,N′-Dicyclohexylcarbodiimide), EDC (1-Ethyl-3-(3-dimethylaminopropyl) carbodiimide), NHS (N-hydroxysuccinimide), Sulfo-NHS (N-hydroxysulfosuccinimide), BMPH, EMCH, KMUH, MPBH, PDPH, and PMPI.

Homobifunctional cross-linking reagents have reactivity toward the same reactive group on a molecule, for example, an antibody. Examples of homobifunctional cross-linking reagents include DTNB (5,5′-dithiobis(2-nitrobenzoic acid), o-PDM (o-phenylenedimaleimide), DMA (dimethyl adipimidate), DMP (dimethyl pimelimidate), DMS (dimethyl suberimidate), DTBP (dithiobispropionimidate), BS(PEG)5, BS(PEG)9, BS3, BSOCOES, DSG, DSP, DSS, DST, DTSSP, EGS, Sulfo-EGS, TSAT, DFDNB, BM(PEG)n crosslinkers, BMB, BMDB, BMH, BMOE, DTME, and TMEA.

Somatic hybridization is the fusion of two distinct hybridoma (a fusion of B cells that produce a specific antibody and myeloma cells) cell lines, producing a quadroma capable of generating two different antibody heavy (V_{H}A and V_{H}B) and light chains (V_{L}A and V_{L}B). (Kontermann, R. E. In: Bispecific Antibodies. Kontermann R E (ed.), Springer Heidelberg Dordrecht London New York, pp. 1-28 (2011)). These heavy and light chains combine randomly within the cell, resulting in bispecific antibodies (a V_{H}A combined with a V_{L}A and a V_{H}B combined with a V_{L}B), as well as some nonfunctional (e.g. two V_{H}As combined with two V_{L}Bs) and monospecific (two V_{H}As combined with two V_{H}As) antibodies. The bispecific antibodies can then be purified using, for example, two different affinity chromatography columns. Similar to monospecific antibodies, bispecific antibodies may also contain an Fc region that elicits Fc-mediated effects downstream of antigen binding. These effects may be reduced by, for example, proteolytically cleaving the Fc region from the bispecific antibody by pepsin digestion, resulting in bispecific F(ab′)₂ molecules (Id.).

Bispecific antibodies may also be generated via genetic means, e.g., in vitro expression of a plasmid containing a DNA sequence corresponding to the desired antibody structure. See, e.g., Kontermann, R. E. In: Bispecific Antibodies. Kontermann R E (ed.), Springer Heidelberg Dordrecht London New York, pp. 1-28 (2011). Such bispecific antibodies are discussed in greater detail below.

A bispecific antibody of the present invention may be bivalent, trivalent, or tetravalent. As used herein, “valent”, “valence”, “valencies”, or other grammatical variations thereof, mean the number of antigen binding sites in an antibody molecule. These antigen recognition sites may recognize the same epitope or different epitopes. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301. Trivalent bispecific antibodies and tetravalent bispecific antibodies are also known in the art. See, e.g., Kontermann R E (ed.), Springer Heidelberg Dordrecht London New York, pp. 199-216 (2011). A bispecific antibody may also have valencies higher than 4 and are also within the scope of the present invention. Such antibodies may be generated by, for example, dock and lock conjugation method. (Chang, C.-H. et al. In: Bispecific Antibodies. Kontermann R E (ed.), Springer Heidelberg Dordrecht London New York, pp. 199-216 (2011)).

The phrase “binds specifically” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antibodies bind to a particular protein sequence, thereby identifying its presence.

Specific binding to an antibody under such conditions requires an antibody that is selected for its specificity for a particular protein. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are well known in the art (see, for example, Harlow and Lane, Antibodies: A laboratory manual (Cold Spring Harbor Laboratory Press, 1988); Friefelder, “Physical Biochemistry: Applications to biochemistry and molecular biology” (W.H. Freeman and Co. 1976)).

In the present invention, an antibody may be characterized by having specific binding activity (Kₐ) for an antigen of at least about 10⁵ mol⁻¹, 10⁶ mol⁻¹ or greater, preferably 10⁷ mol⁻¹ or greater, more preferably 10⁸ mol⁻¹ or greater, and most preferably 10⁹ mol⁻¹ or greater. The binding affinity of an antibody can be readily determined by one of ordinary skill in the art, for example, by Scatchard analysis (Scatchard, Ann. NY Acad. Sci. 51: 660-72, 1949).

As used herein, the term “antigen binding moiety” refers to the regions of a polypeptide molecule that specifically bind to an antigen. Non-limiting examples of antigen binding moieties include immunoglobulins and derivatives such as Fv, Fab, Fab′, Fab′-SH, F(ab′)₂.

In one aspect of an embodiment of the present invention, the first antigen binding moiety specifically binds an epitope in the extracellular IgV domain of the human CTLA-4.

CTLA-4 is a T-cell surface molecule that is purported to be involved in the down-regulation of the immune response. CTLA-4 contains an extracellular IgV domain, a transmembrane domain, and a short cytoplasmic tail. The extracellular IgV domain of the human CTLA-4 protein is the first 125 amino acids of the full length human CTLA-4 protein (Dariavach, 1988).

In another aspect of this embodiment, the second antigen binding moiety specifically binds an epitope in the extracellular IgV domain of the human PD-1 receptor. PD-1 is related to CTLA-4 and also has a extracellular IgV domain, a transmembrane domain, and a short cytoplasmic tail. The extracellular IgV domain of the human PD-1 protein is the first 167 amino acids of the full length human PD-1 protein (Shinohara et al., 1994).

In a further aspect of this embodiment, the bispecific antibody is a recombinant antibody, a monoclonal antibody, a chimeric antibody, a humanized antibody, a human antibody, or an antibody fragment.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), and as modified by the somatic hybridization method as set forth above; or may be made by other recombinant DNA methods (see, e.g., U.S. Pat. No. 4,816,567).

Additional types of antibodies that may be part of the bispecific antibodies of the present invention include, but are not limited to, chimeric, humanized, and human antibodies. For application in man, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g. a variable domain) derived from one species (e.g. mouse) fused to a domain (e.g. the constant domains) derived from a different species (e.g. human).

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol 2:593-596 (1992)). Humanization can be essentially performed following the method of Winter and co-workers (Jones et al., Nature 321:522-525 (1986); Riechmann et al., Nature 332:323-3′27 (1988); Verhoeyen et al., Science 239:1534-1536 (1988)), by substituting rodent CDRs or CDR sequences for the corresponding sequences of a human antibody.

Furthermore, technologies have been developed for creating antibodies based on sequences derived from the human genome, for example by phage display or using transgenic animals (WO 90/05144; D. Marks, H. R. Hoogenboom, T. P. Bonnert, J. McCafferty, A. D. Griffiths and G. Winter (1991) “By-passing immunisation. Human antibodies from V-gene libraries displayed on phage.” J. Mol. Biol., 222, 581-597; Knappik et al., J. Mol. Biol. 296: 57-86, 2000; S. Carmen and L. Jermutus, “Concepts in antibody phage display”. Briefings in Functional Genomics and Proteomics 2002 1(2):189-203; Lonberg N, Huszar D. “Human antibodies from transgenic mice”. Int Rev Immunol. 1995; 13(1):65-93.; Bruggemann M, Taussig M J. “Production of human antibody repertoires in transgenic mice”. Curr Opin Biotechnol. 1997 August; 8(4):455-8.). Such antibodies are “human antibodies” in the context of the present invention.

As used herein, “recombinant” antibody means any antibody whose production involves expression of a non-native DNA sequence encoding the desired antibody structure in an organism. In the present invention, recombinant antibodies include tandem scFv (taFv or scFv₂), diabody, dAb₂/VHH₂, knob-into-holes derivates, SEED-IgG, heteroFc-scFv, Fab-scFv, scFv-Jun/Fos, Fab′-Jun/Fos, tribody, DNL-F(ab)₃, scFv₃-CH1/CL, Fab-scFv₂, IgG-scFab, IgG-scFv, scFv-IgG, scFv₂-Fc, F(ab′)₂-scFv₂, scDB-Fc, scDb-CH3, Db-Fc, scFv₂-H/L, DVD-Ig, tandAb, scFv-dhlx-scFv, dAb₂-IgG, dAb-IgG, dAb-Fc-dAb, and combinations thereof.

Variable regions of antibodies are typically isolated as single-chain Fv (scFv) or Fab fragments. ScFv fragments are composed of V_{H} and V_{L} domains linked by a short 10-25 amino acid linker. Once isolated, scFv fragments can be genetically linked with a flexible peptide linker such as, for example, one or more repeats of Ala-Ala-Ala, Gly-Gly-Gly-Gly-Ser, etc. The resultant peptide, a tandem scFv (taFv or scFv₂) can be arranged in various ways, with V_{H}-V_{L} or V_{L}-V_{H} ordering for each scFv of the taFv. (Kontermann, R. E. In: Bispecific Antibodies. Kontermann R E (ed.), Springer Heidelberg Dordrecht London New York, pp. 1-28 (2011)).

Bispecific diabodies are another form of antibody fragment and are within the scope of the present invention. In contrast to taFvs, diabodies are composed of two separate polypeptide chains from, for example, antibodies A and B, each chain bearing two variable domains (V_{H}A-V_{L}B and V_{H}B-V_{L}A or V_{L}A-V_{H}B and V_{L}B-V_{H}A). The linkers joining the variable domains are short (about five amino acids), preventing the association of V_{H} and V_{L} domains on the same chain, and promoting the association of V_{H} and V_{L} domains on different chains. Heterodimers that form are functional against both target antigens, (such as, e.g., V_{H}A-V_{L}B with V_{H}B-V_{L}A or V_{L}A-V_{H}B with V_{L}B-V_{H}A), however, homodimers can also form (such as, e.g., V_{H}A-V_{L}B with V_{H}A-V_{L}B, V_{H}B-V_{L}A with V_{H}B-V_{L}A, etc.), leading to nonfunctional molecules. Several strategies exist to prevent homodimerization, including the introduction of disulfide bonds to covalently join the two polypeptide chains, modification of the polypeptide chains to include large amino acids on one chain and small amino acids on the other (knobs-into-holes structures, discussed below), and addition of cysteine residues at C-terminal extensions. Another strategy is to join the two polypeptide chains by a linker sequence, producing a single-chain diabody molecule (scDb) that exhibits a more compact structure than a taFv. ScDbs or Dbs can be also be fused to the IgG1 C_{H}3 domain or the Fc region, producing di-diabodies. Examples of di-diabodies include, but are not limited to, scDb-Fc, Db-Fc, scDb-C_{H}3, and Db-C_{H}3. Additionally, scDbs can be used to make tetravalent bispecific molecules. By shortening the linker sequence of scDbs from about 15 amino acids to about 5 amino acids, dimeric single-chain diabody molecules result, known as TandAbs (Muller, D. and Kontermann, R. E. In: Bispecific Antibodies. Kontermann R E (ed.), Springer Heidelberg Dordrecht London New York, pp. 83-100 (2011)).

Yet another strategy for generating a bispecific antibody according to the present invention includes fusing heterodimerizing peptides to the C-termini of the antibody molecules (scFvs or Fabs). A non-limiting example of this strategy is the use of antibody fragments linked to jun-fos leucine zippers (e.g. scFv-Jun/Fos and Fab′-Jun/Fos).

An additional method according to the present invention for generating a bispecific antibody molecule includes derivatizing two antibodies with different antigen binding moieties with biotin and then linking the two antibodies via strepavidin, followed by purification and isolation of the resultant bispecific antibody.

In the present invention, constant immunoglobulin domains can also be used to promote heterodimerization of two polypeptide chains (IgG-like antibodies, discussed below). Non-limiting examples of this type of approach to making a bispecific antibody include the introduction of knobs-into-holes structures into the two polypeptides and utilization of the naturally occurring heterodimerization of the C_{L} and C_{H}1 domains (Kontermann, R. E. In: Bispecific Antibodies. Kontermann R E (ed.), Springer Heidelberg Dordrecht London New York, pp. 1-28 (2011)).

Additional types of bispecific antibodies according to the present invention include those that contain more than one antigen-binding site for each antigen. For example, additional V_{H} and V_{L} domains can be fused to the N-terminus of the V_{H} and V_{L} domains of an existing antibody, effectively arranging the antigen-binding sites in tandem. These types of antibodies are known as dual-variable-domain antibodies (DVD-Ig) (Tarcsa, E. et al. In: Bispecific Antibodies. Kontermann R E (ed.), Springer Heidelberg Dordrecht London New York, pp. 171-185 (2011)). Yet another method according to the present invention for producing antibodies that contain more than one antigen-binding site for an antigen is to fuse scFv fragments to the N-terminus of the heavy chain or the C-terminus of the light chain (discussed further below).

The majority of the recombinant antibody types according to the present invention can be engineered to be IgG-like, meaning that they also include an Fc domain. Similar to diabodies that require heterodimerization of engineered polypeptide chains, IgG-like antibodies also require heterodimerization to prevent the interaction of like heavy chains or heavy chains and light chains from two antibodies of different specificity (Jin, P. and Zhu, Z. In: Bispecific Antibodies. Kontermann RE (ed.), Springer Heidelberg Dordrecht London New York, pp. 151-169 (2011)).

Knobs-into-holes structures facilitate heterodimerization of polypeptide chains by introducing large amino acids (knobs) into one chain of a desired heterodimer and small amino acids (holes) into the other chain of the desired heterodimer. Steric interactions will favor the interaction of the knobs with holes, rather than knobs with knobs or holes with holes. In the context of bispecific IgG-like antibodies, like heavy chains can be prevented from homodimerizing by the introduction of knobs-into-holes structures into the C_{H}3 domain of the Fc region. Similarly, promoting the interaction of heavy chains and light chains specific to the same antigen can be accomplished by engineering knobs-into-holes structures at the V_{H}-V_{L} interface. Other examples of knobs-into-holes structures exist and the examples discussed above should not be construed to be limiting. Other methods to promote heterodimerization of Fc regions include engineering charge polarity into Fc domains (Gunasekaran et al., 2010) and SEED technology (SEED-IgG) (Davis et al., 2010).

Additional heterodimerized IgG-like antibodies include, but are not limited to, heteroFc-scFvs, Fab-scFvs, IgG-scFv, and scFv-IgG. HeteroFc-scFvs link two distinct scFvs to heterodimerizable Fc domains while Fab-scFvs contain an Fab domain specific to one epitope linked to an scFv specific to a different epitope. IgG-scFv and scFv-IgG are Ig-like antibodies that have scFvs linked to their C-termini and N-termini, respectively (Kontermann, R. E. In: Bispecific Antibodies. Kontermann R E (ed.), Springer Heidelberg Dordrecht London New York, pp. 151-169 (2011)).

Though most naturally occurring antibodies are composed of heavy chains and light chains, camelids (e.g. camels, dromedaries, llamas, and alpacas) and some sharks produce antibodies that consist only of heavy chains. These antibodies bind antigenic epitopes using a single variable domain known as VHH. When produced in Escherichia coli, these molecules are termed single domain antibodies (dAbs). The simplest application of dAbs in bispecific antibodies is to link two different dAbs together to form dAb₂s (VHH₂s). dAbs can also be applied to IgG-like bispecific antibodies. Examples of this include, but are not limited to, dAb₂-IgGs, dAb-IgGs, and dAb-Fc-dAbs. dAb₂-IgGs have a similar structure to intact antibodies, but with dAbs linked to the N-terminal end of the molecule. dAb-IgGs are intact antibodies specific for one epitope with a single dAb specific for another epitope linked to the N-termini or C-termini of the heavy chains. Lastly, dAb-Fc-dAbs are Fc domains with dAbs specific for one epitope linked to the N-termini and dAbs specific for another epitope linked to the C-termini (Chames, P. and Baty, D. In: Bispecific Antibodies. Kontermann R E (ed.), Springer Heidelberg Dordrecht London New York, pp. 101-114 (2011)). Each of the foregoing antibodies is within the scope of the present invention.

Several types of trivalent antibodies have been developed. Triplebodies are composed of three distinct scFv regions joined by linker sequences approximately 20 amino acids in length. Tribodies utilize the natural in vivo heterodimerization of the heavy chain (C_{H}1 domain) and light chain (C_{L} domain) to form a scaffold on which multiple scFvs can be added. For example, a scFv specific to one antigen can be linked to a C_{H}1 domain, which is also linked to a scFv specific to another antigen and this chain can interact with another chain containing an scFv specific to either antigen linked to a C_{L} domain (scFv₃-C_{H}1/C_{L}). Another example of a trivalent construction involves the use of a Fab fragment specific to one epitope C-terminally linked to two scFvs specific to another epitope, one on each chain (Fab-scFv₂). Yet another example of a trivalent molecule consists of an intact antibody molecule specific to one antigen with a single chain Fab (scFab) linked to the C-terminal end of the molecule (IgG-scFab). The dock-and-lock (DNL) approach has also been used to generate trivalent antibodies (DNL-F(ab)₃) (Chang, C.-H. et al. In: Bispecific Antibodies. Kontermann R E (ed.), Springer Heidelberg Dordrecht London New York, pp. 199-216 (2011)). Each of the foregoing antibodies is within the scope of the present invention.

Tetravalent antibodies have also been constructed. Examples of tetravalent antibodies include, but are not limited to, scFv₂-Fc, F(ab′)₂-scFv₂, scFv₂-H/L, and scFv-dhlx-scFv molecules. Bispecific scFv₂-Fc constructs have an Fc domain with two scFvs specific to one molecule linked to the N-termini of the Fc chains and another two scFvs specific to another molecule linked to the C-termini of the Fc chain. Bispecific F(ab′)₂-scFv₂ constructs include scFv fragments linked to the C-terminal end of an F(ab′)₂ fragment. scFv₂-H/L constructs have scFvs specific to one molecule linked to the heavy chains while scFvs specific to another molecule are linked to the light chains. Finally, scFv-dhlx-scFv constructs contain one type of scFv linked to a helical dimerization domain followed by another type of scFv. Two chains of this type can dimerize, generating a tetravalent antibody (Kontermann, R. E. In: Bispecific Antibodies. Kontermann R E (ed.), Springer Heidelberg Dordrecht London New York, pp. 1-28 (2011)). Each of the foregoing antibodies is within the scope of the present invention.

Variable regions of antibodies are typically isolated as single-chain Fv (scFv) or Fab fragments. ScFv fragments are composed of V_{H} and V_{L} domains linked by a short 10-25 amino acid linker. Once isolated, scFv fragments can be genetically linked with a flexible peptide linker such as, for example, one or more repeats of Ala-Ala-Ala, Gly-Gly-Gly-Gly-Ser, etc. The resultant peptide, a tandem scFv (taFv or scFv₂) can be arranged in various ways, with V_{H}-V_{L} or V_{L}-V_{H} ordering for each scFv of the taFv (Id.). Each of these constructs may be used, as appropriate in the present invention.

In another aspect of this embodiment, the first antigen binding moiety comprises a variable heavy chain as depicted in SEQ ID NO:5, a variable light chain as depicted in SEQ ID NO:6 and the second antigen binding moiety comprises a variable heavy chain as depicted in SEQ ID NO:11, a variable light chain as depicted in SEQ ID NO:12.

In an additional aspect of this embodiment, first and second first antigen binding moieties are connected directly or by a linker.

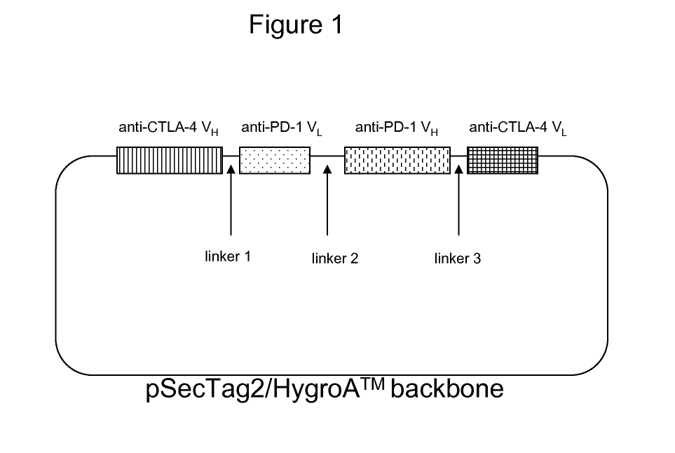

In this context, the term “linker” refers to any means that serves to join two distinct functional units (e.g. antigen binding moieties). Types of linkers include, but are not limited to, chemical linkers and polypeptide linkers. Various types of chemical linkers are as set forth above. The sequences of the polypeptide linkers are not limited. Polypeptide linkers are preferably non-immunogenic and flexible, such as those comprising serine and glycine sequences or repeats of Ala-Ala-Ala. Depending on the particular construct, the linkers may be long or short. For example, to make a single chain diabody as set forth in FIG. 1, the first and the third linkers are preferably 3-12 residues, more preferably about 5 amino acids in length, and the second linker is preferably longer than 12 residues, and more preferably about 15 amino acids in length. Reducing the linker length to below 3 residues can force single chain antibody fragments into trimers or tetramers. (Hudson et al., 1999). Thus, in the present invention the bispecific antibody may be, e.g., bivalent, trivalent or tetravalent, as desired.

Another embodiment of the present invention is a bispecific antibody. This bispecific antibody comprises:

(a) a first antigen binding moiety that specifically binds an epitope on human cytotoxic T-lymphocyte-associated antigen 4 (CTLA-4), and

(b) a second antigen binding moiety that specifically binds an epitope on a human programmed death 1 (PD-1) receptor.

The preferred anti-CTLA-4 antibody is a human antibody that specifically binds to human CTLA-4. Exemplary human anti-CTLA-4 antibodies are described in detail in International Application No. PCT/US99/30895, published on Jun. 29, 2000 as WO 00/37504, European Patent Appl. No. EP 1262193 A1, published Apr. 12, 2002, and U.S. patent application Ser. No. 09/472,087, now issued as U.S. Pat. No. 6,682,736, to Hanson et al., as well as U.S. patent application Ser. No. 09/948,939, published as US2002/0086014. Such antibodies include, but are not limited to, 3.1.1, 4.1.1, 4.8.1, 4.10.2, 4.13.1, 4.14.3, 6.1.1, tremelimumab (formerly ticilimumab, CP-675,206, manufactured by Pfizer, New York, N.Y.), 11.6.1, 11.7.1, 12.3.1.1, and 12.9.1.1, as well as ipilimumab (also known as Yervoy®, MDX-010 and MDX-101 manufactured by Bristol-Myers Squibb Company. Princeton, N.J.) and other human anti-CTLA-4 antibodies disclosed in U.S. patent application Ser. No. 09/948,939, published as U.S. Patent Application Publication No. 2002/0086014 and No. 2003/0086930. The entire contents of the above patents and patent applications, including all of the amino and nucleic acid sequences set forth therein, are incorporated by reference, as if fully recited herein.

Characteristics of useful human anti-CTLA-4 antibodies of the invention are extensively discussed in WO 00/37504, EP 1262193, and U.S. Pat. No. 6,682,736 as well as U.S. Patent Application Publication Nos. US2002/0086014 and US2003/0086930. Briefly, the antibodies of the invention include antibodies having amino acid sequences of the heavy and light chains of an antibody such as, but not limited to, antibody 3.1.1, 4.1.1, 4.8.1, 4.10.2, 4.13.1, 4.14.3, 6.1.1, tremelimumab, 11.6.1, 11.7.1, 12.3.1.1, 12.9.1.1, and ipilimumab. The invention also relates to antibodies having the amino acid sequences of the CDRs of the heavy and light chains of these antibodies, as well as those having changes in the CDR regions, as described in the above-cited applications and patent. The present invention also includes antibodies having the variable regions of the heavy and light chains of those antibodies.

The preferred anti-PD-1 antibody is a human antibody that specifically binds to human PD-1. Exemplary human anti-PD-1 antibodies include nivolumab from Bristol-Myers Squibb Company (CAS Registry No. 946414-94-4, also known as MDX-1106, BMS-936558, or ONO-4538) (fully human IgG4 anti-PD1 mAb), CT-011 (humanized IgG1 anti-PD1 mAb from CureTech Ltd., Yavne, Israel and Teva Pharmaceutical Industries, Ltd., Petach Tikva, Israel), lambrolizumab (also known as MK-3475) (human IgG4 anti-PD1 mAb from Merck, Whitehouse Station, N.J.), and AMP-224 (a B7-DC/IgG1 fusion protein licensed to GlaxoSmithKline plc (GSK), Philadelphia, Pa.), and other human monoclonal antibodies disclosed in U.S. Pat. No. 8,008,449 issued on Aug. 30, 2011, and in U.S. Patent Publication No. 20090263386. The entire contents of the above patents and patent applications, including all of the amino and nucleic acid sequences set forth therein, are incorporated by reference, as if fully recited herein.

In one aspect of this embodiment, the first antigen binding moiety specifically binds an epitope in the extracellular IgV domain of the human CTLA-4.

In another aspect of this embodiment, the second antigen binding moiety specifically binds an epitope in the extracellular IgV domain of the human PD-1 receptor.

In a further aspect of this embodiment, the bispecific antibody is a recombinant antibody, a monoclonal antibody, a chimeric antibody, a humanized antibody, a human antibody, or an antibody fragment.

In an additional aspect of this embodiment, first and second antigen binding moieties are connected directly or by a linker such as, e.g., a chemical or polypeptide linker. Suitable and preferred linkers are as set forth above.

In another aspect of this embodiment, the first antigen binding moiety comprises a variable heavy chain and a variable light chain of ipilimumab, and the second antigen binding moiety comprises a variable heavy chain and a variable light chain of nivolumab.

In an additional aspect of this embodiment, the first antigen binding moiety comprises a variable heavy chain and a variable light chain of tremelimumab, and the second antigen binding moiety comprises a variable heavy chain and a variable light chain of nivolumab.

In a further aspect of this embodiment, each antigen binding moiety is independently selected from the group consisting of IgM, IgG, IgD, IgA, IgE, antibody fragments that retain antigen recognition and binding capability that are Fab, Fab′, F(ab′)₂, and Fv fragments, and combinations thereof, and further wherein the first and second antigen binding moieties are connected directly or by a linker.

In another aspect of this embodiment, the bispecific antibody is bivalent, trivalent, or tetravalent. Preferably, the bispecific antibody is selected from the group consisting of a tandem scFv (taFv or scFv₂), diabody, dAb₂/VHH₂, knob-into-holes derivates, SEED-IgG, heteroFc-scFv, Fab-scFv, scFv-Jun/Fos, Fab′-Jun/Fos, tribody, DNL-F(ab)₃, scFv₃-CH1/CL, Fab-scFv₂, IgG-scFab, IgG-scFv, scFv-IgG, scFv₂-Fc, F(ab′)₂-scFv₂, scDB-Fc, scDb-CH3, Db-Fc, scFv₂-H/L, DVD-Ig, tandAb, scFv-dhlx-scFv, dAb₂-IgG, dAb-IgG, dAb-Fc-dAb, and combinations thereof. More preferably, the bispecific antibody is a diabody or a tribody.

An additional embodiment of the present invention is a pharmaceutical composition. This pharmaceutical composition comprises any bispecific antibody disclosed herein and a pharmaceutically acceptable excipient.

A further embodiment of the present invention is a method of treating cancer in a subject. This method comprises administering to the subject a therapeutically effective amount of any pharmaceutical composition disclosed herein.

As used herein, a “subject” is a mammal, preferably, a human. In addition to humans, categories of mammals within the scope of the present invention include, for example, agricultural animals, domestic animals, laboratory animals, etc. Some examples of agricultural animals include cows, pigs, horses, goats, etc. Some examples of domestic animals include dogs, cats, etc. Some examples of laboratory animals include rats, mice, rabbits, guinea pigs, etc.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

Nonlimiting examples of cancers that may be treated in accordance with the present invention include adrenocortical carcinoma, anal tumor/cancer, bladder tumor/cancer, bone tumor/cancer (such as osteosarcoma), brain tumor, breast tumor/cancer, carcinoid tumor, carcinoma, cervical tumor/cancer, colon tumor/cancer, endometrial tumor/cancer, esophageal tumor/cancer, extrahepatic bile duct tumor/cancer, Ewing family of tumors, extracranial germ cell tumor, eye tumor/cancer, gallbladder tumor/cancer, gastric tumor/cancer, germ cell tumor, gestational trophoblastic tumor, head and neck tumor/cancer, hypopharyngeal tumor/cancer, islet cell carcinoma, kidney tumor/cancer, laryngeal tumor/cancer, leukemia, lip and oral cavity tumor/cancer, liver tumor/cancer, lung tumor/cancer, lymphoma, malignant mesothelioma, Merkel cell carcinoma, mycosis fungoides, myelodysplastic syndrome, myeloproliferative disorders, nasopharyngeal tumor/cancer, neuroblastoma, oral tumor/cancer, oropharyngeal tumor/cancer, ovarian epithelial tumor/cancer, ovarian germ cell tumor, pancreatic tumor/cancer, paranasal sinus and nasal cavity tumor/cancer, parathyroid tumor/cancer, penile tumor/cancer, pituitary tumor/cancer, plasma cell neoplasm, prostate tumor/cancer, rhabdomyosarcoma, rectal tumor/cancer, renal cell tumor/cancer, transitional cell tumor/cancer of the renal pelvis and ureter, salivary gland tumor/cancer, Sezary syndrome, skin tumors (such as cutaneous t-cell lymphoma, Kaposi's sarcoma, mast cell tumor, and melanoma), small intestine tumor/cancer, soft tissue sarcoma (such as fibrosarcoma), stomach tumor/cancer, testicular tumor/cancer, thymoma, thyroid tumor/cancer, urethral tumor/cancer, uterine tumor/cancer, vaginal tumor/cancer, vulvar tumor/cancer, and Wilms' tumor. Cancers also include liquid tumors such as those in the bone marrow, the blood, or the lymph nodes.

Preferably, the cancer is selected from the group consisting of melanoma, lung cancer, and renal cancer. More preferably, the cancer is melanoma.

In the present invention, an “effective amount” or a “therapeutically effective amount” of a bispecific antibody or a pharmaceutical composition disclosed herein is an amount of such antibody or pharmaceutical composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a composition according to the invention will be that amount of the composition, which is the lowest dose effective to produce the desired effect. The effective dose of a compound or composition of the present invention may be administered as two, three, four, five, six or more sub-doses, administered separately at appropriate intervals throughout the day.

A suitable, non-limiting example of a dosage of a bispecific antibody according to the present invention is from about 0.1 mg/kg to about 20 mg/kg per day, such as from about 0.3 mg/kg to about 10 mg/kg per day, including from about 0.3 mg/kg to about 2.5 mg/kg per day and about 1 mg/kg per day. Other representative dosages of such agents include about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, and 20 mg/kg per day. The effective dose of bispecific antibody disclosed herein may be administered as two, three, four, five, six or more sub-doses, administered separately at appropriate intervals throughout the day.

In any of the methods of treatment disclosed herein, the method may further comprise administering to the subject a therapeutically effective amount of another anti-cancer agent, such as the monospecific antibodies disclosed herein, e.g., ipilimumab, tremelimumab, and nivolumab. In the present invention, the bispecific antibody and the additional anti-cancer agent may be co-administered together in the same composition, simultaneously in separate compositions, or as separate compositions administered at different times, as deemed most appropriate by a physician.

A suitable, non-limiting example of a dosage of monospecific antibody disclosed herein is from about 0.1 mg/kg to about 20 mg/kg per day, such as from about 0.3 mg/kg to about 10 mg/kg per day, including from about 0.3 mg/kg to about 2.5 mg/kg per day and about 1-2 mg/kg per day. Other representative dosages of such agents include about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, and 20 mg/kg per day. The effective dose of the monospecific antibody may be administered as two, three, four, five, six or more sub-doses, administered separately at appropriate intervals throughout the day.

Another embodiment of the present invention is a method of treating cancer in a subject. This method comprises administering to the subject a therapeutically effective amount of a bispecific antibody one antigen binding moiety of which specifically binds human CTLA-4 and the other antigen binding moiety of which binds to human PD-1 receptor. Suitable and preferred bispecific antibodies, types of cancers, and subjects for this embodiment are as set forth above.

An additional embodiment of the present invention is a method of treating melanoma in a subject. This method comprises administering to the subject a therapeutically effective amount of at least one isolated bispecific antibody comprising a first antigen binding moiety that specifically binds an epitope in the extracellular Ig V domain of the human CTLA-4 and a second antigen binding moiety that specifically binds an epitope in the extracellular Ig V domain of the human PD-1 receptor.

In one aspect of this embodiment, the first antigen binding moiety comprises a heavy chain and a light chain of ipilimumab and the second antigen binding moiety comprises a heavy chain and a light chain of nivolumab. Additional suitable and preferred bispecific antibodies and subjects for this embodiment are as set forth above. In this embodiment, from about 0.3-10 mg/kg of the bispecific antibody is administered to the subject, such as for example from about 0.3-2.5 mg/kg or less than about 1 mg/kg of the bispecific antibody.

In another aspect of this embodiment, the method further comprising administering to the subject a therapeutically effective amount of an ipilimumab. Preferably, about 0.3-1 mg/kg of the bispecific antibody and about 1-2 mg/kg of the ipilimumab is administered to the subject.

A further embodiment of the present invention is a kit for treating a cancer in a subject. This kit comprise any pharmaceutical composition disclosed herein.

For use in the kits of the invention, pharmaceutical compositions comprising suitable and preferred bispecific antibodies, types of cancers, and subjects are as set forth above. The kits may also include suitable storage containers, e.g., ampules, vials, tubes, etc., for each pharmaceutical composition and other included reagents, e.g., buffers, balanced salt solutions, etc., for use in administering the pharmaceutical compositions to subjects. The pharmaceutical compositions and other reagents may be present in the kits in any convenient form, such as, e.g., in a solution or in a powder form. The kits may further include instructions for use of the pharmaceutical compositions. The kits may further include a packaging container, optionally having one or more partitions for housing the pharmaceutical composition and other optional reagents.

Another embodiment of the present invention is a bispecific antibody. This antibody comprises:

(a) a first antigen binding moiety that specifically binds an epitope on human cytotoxic T-lymphocyte-associated antigen 4 (CTLA-4), and

(b) a second antigen binding moiety that specifically binds an epitope on a human programmed death ligand 1 (PD-L1).

Suitable and preferred first antigen binding moieties are as set forth above.

The preferred anti-PD-L1 antibody is a human antibody that specifically binds to human PD-L1. Exemplary human anti-PD-1 antibodies include MPDL3280A/RG7446 (an anti-PD-L1 antibody manufactured by Genentech, San Francisco, Calif.). Other exemplary antibodies are disclosed in U.S. Pat. No. 8,217,149 issued on Jul. 10, 2012, and U.S. Pat. No. 7,943,743 issued on May 17, 2011. The entire contents of the above patents, including all of amino and nucleic acid sequences set forth therein, are incorporated by reference, as if fully recited herein.

Receptor fusion proteins, in which the receptor is fused to Fc region of an IgG molecule, are also contemplated in this embodiment. For example, suitable CTLA-4 fusion proteins are disclosed in WO1993000431 A1. PD-1-Fc fusion proteins are also known in the art and are commercially available from R&D Systems (Minneapolis, Minn.). Chimeric receptor-Fc fusion proteins may be made polymeric using methods disclosed in Mekhaiel et al., 2011, or using other methods herein.

In one aspect of this embodiment, the bispecific antibody is a recombinant antibody, a monoclonal antibody, a chimeric antibody, a humanized antibody, a human antibody, or an antibody fragment.

In another aspect of this embodiment, first and second first antigen binding moieties are connected directly or by a linker, such as a chemical linker or a polypeptide linker. Suitable and preferred linkers are as disclosed herein.

In an additional aspect of this embodiment, each antigen binding moiety is independently selected from the group consisting of IgM, IgG, IgD, IgA, IgE, antibody fragments that retain antigen recognition and binding capability that are Fab, Fab′, F(ab′)₂, and Fv fragments, and combinations thereof, and further wherein the first and second antigen binding moieties are connected directly or by a linker.

In a further aspect of this embodiment, the bispecific antibody is bivalent, trivalent, or tetravalent.

In another aspect of this embodiment, the bispecific antibody is selected from the group consisting of a tandem scFv (taFv or scFv₂), diabody, dAb₂/VHH₂, knob-into-holes derivates, SEED-IgG, heteroFc-scFv, Fab-scFv, scFv-Jun/Fos, Fab′-Jun/Fos, tribody, DNL-F(ab)₃, scFv₃-CH1/CL, Fab-scFv₂, IgG-scFab, IgG-scFv, scFv-IgG, scFv₂-Fc, F(ab′)₂-scFv₂, scDB-Fc, scDb-CH3, Db-Fc, scFv₂-H/L, DVD-Ig, tandAb, scFv-dhlx-scFv, dAb₂-IgG, dAb-IgG, dAb-Fc-dAb, and combinations thereof.

An additional embodiment of the present invention is a method of treating cancer in a subject. This method comprises administering to the subject a therapeutically effective amount of a bispecific antibody, one antigen binding moiety of which specifically binds human CTLA-4 and the other antigen binding moiety of which binds to human PD-L1.

Suitable and preferred cancers, subject, bispecific antibody, and effective amounts thereof are set forth above.

An additional embodiment of the present invention is a method for preventing cancer. This method comprise comprises administering to the subject a therapeutically effective amount of a cancer vaccine and at least one isolated bispecific antibody disclosed herein.

As used herein, the terms “prevent”, “preventing” and grammatical variations thereof mean to administer a compound or a composition of the present invention to a subject who has not been diagnosed as having the disease or condition at the time of administration, but who could be expected to develop the disease or condition or be at increased risk for the disease or condition. Preventing also includes administration of at least one compound or a composition of the present invention to those subjects thought to be predisposed to the disease or condition due to age, familial history, genetic or chromosomal abnormalities, due to the presence of one or more biological markers for the disease or condition and/or due to environmental factors.

Suitable and preferred bispecific antibodies, types of cancers, and subjects for this embodiment are as set forth above. Cancer vaccines include, without limitation, GVAX vaccination (granulocyte macrophage colony-stimulating factor-expressing irradiated tumor cells) and FVAX (Flt3-ligand). (Curran et al., 2011; Curran et al., 2010; Duraiswamy et al., 2013).

A further embodiment of the present invention is a method for treating the Human Immunodeficiency Virus (HIV). This method comprise comprises administering to the subject a therapeutically effective amount of at least one isolated bispecific antibody disclosed herein.

Suitable and preferred bispecific antibodies and subjects for this embodiment are as set forth above.

As used herein, terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymers.

The term “amino acid” means naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. An “amino acid analog” means compounds that have the same basic chemical structure as a naturally occurring amino acid, e.g., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. An “amino acid mimetic” means a chemical compound that has a structure that is different from the general chemical structure of an amino acid, but that functions similarly to a naturally occurring amino acid.

A bispecific antibody or a pharmaceutical composition of the present invention may be administered to a subject in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a pharmaceutical composition of the present invention may be administered in conjunction with other treatments, as set forth above. A pharmaceutical composition of the present invention may be encapsulated or otherwise protected against gastric or other secretions, if desired.

The pharmaceutical compositions of the invention may comprise one or more active ingredients in admixture with one or more pharmaceutically-acceptable carriers and, optionally, one or more other compounds, drugs, ingredients and/or materials. Regardless of the route of administration selected, the bispecific antibodies of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions of the invention may, optionally, contain additional ingredients and/or materials commonly used in pharmaceutical compositions, including therapeutic antibody preparations. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions of the present invention for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions of the present invention which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier. The ointments, pastes, creams and gels may contain excipients. Powders and sprays may contain excipients and propellants.

Pharmaceutical compositions of the present invention suitable for parenteral administrations comprise one or more agent(s)/compound(s)/antibodies in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug/antibody then depends upon its rate of dissolution which, in turn, may depend upon crystal size and crystalline form. Alternatively, delayed absorption of a parenterally-administered agent/drug/antibody may be accomplished by dissolving or suspending the active agent/drug/antibody in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The following examples are provided to further illustrate the methods of the present invention. These examples are illustrative only and are not intended to limit the scope of the invention in any way.

### EXAMPLES

### Example 1

### Making of anti-PD-1 and anti-CTLA-4 bispecific antibodies

### Recombinant DNA Techniques

Standard methods are used to manipulate DNA as described in Sambrook et al., Molecular cloning: A laboratory manual; Cold Spring Harbor Laboratory Press, Cold Spring Harbor, N. Y., 1989. The molecular biological reagents are used according to the manufacturers' instructions. General information regarding the nucleotide sequences of human immunoglobulins light and heavy chains is given in: Kabat, E. A. et al., (1991) Sequences of Proteins of Immunological Interest, 5th ed., NIH Publication No. 91-3242.

### Gene Synthesis

Desired gene segments are either generated by PCR using appropriate templates or are synthesized from synthetic oligonucleotides and PCR products by automated gene synthesis. Such gene synthesis is commercially available from, e.g., Invitrogen (Life Technologies, Inc. Carlsbad, Calif.) and Geneart AG (Regensburg, Germany). The gene segments flanked by singular restriction endonuclease cleavage sites are cloned into standard cloning/sequencing vectors. The plasmid DNA is purified from transformed bacteria and concentration determined by UV spectroscopy. The DNA sequence of the subcloned gene fragments is confirmed by DNA sequencing. Gene segments are designed with suitable restriction sites to allow sub-cloning into the respective expression vectors.

### DNA Constructs-Diabodies

DNAs encoding bispecific single chain diabodies are constructed as follows as shown in FIG. 1. Specifically, the variable heavy chain of a CTLA-4 antibody (either ipilimumab or tremelimumab) is linked via a five amino acid linker (linker 1) to the variable light chain a anti-human PD-1 antibody, which, in turn, is linked via a fifteen amino acid linker (linker 2) to the variable heavy chain of the anti-human PD-1 antibody, which is linked via another five amino acid linker (linker 3) to the variable light chain of the CTLA-4 antibody.

In the first construct for a bispecific single chain diabody, the variable heavy chain of a CTLA-4 antibody (ipilimumab, the amino acid sequence of which is listed in SEQ ID NO:1) is linked via linker 1 (SGGGG, SEQ ID NO:13), to the variable light chain of an anti-human PD-1 antibody (the amino acid sequence of which is listed in SEQ ID NO:10), which, in turn, is linked via linker 2 (SGGGGSGGGGSGGGG, SEQ ID NO:14) to the variable heavy chain of the anti-human PD-1 antibody (SEQ ID NO:9), followed by linker 3 (SGGGG, SEQ ID NO:15) and the variable light chain of anti-human CTLA-4 antibody (ipilimumab, the amino acid sequence of which is listed in SEQ ID NO:2). The resulting single chain diabody is referred to the “ipilimumab-PD-1” diabody below.

In the second construct for a bispecific single chain diabody, the variable heavy chain of another CTLA-4 antibody (tremelimumab, the amino acid sequence of which is listed in SEQ ID NO:5) is linked via linker 1 (SGGGG, SEQ ID NO:13), to the variable light chain of an anti-human PD-1 antibody (the amino acid sequence of which is listed in SEQ ID NO:10), which, in turn, is linked via linker 2 (SGGGGSGGGGSGGGG, SEQ ID NO:14) to the variable heavy chain of the anti-human PD-1 antibody (SEQ ID NO:9), followed by linker 3 (SGGGG, SEQ ID NO:15) and the variable light chain of anti-human CTLA-4 antibody (ipilimumab, the amino acid sequence of which is listed in SEQ ID NO:6). The resulting single chain diabody is referred to the “tremelimumab-PD-1” diabody below.

DNA encoding each of the two bispecific diabodies is separately cloned into expression vector pSecTag2/HygroA (Invitrogen, Life Technologies). The resulting plasmid encoding the bispecific antibody (pSecTag2/HygroA-PD1-CTLA-4-ipi or pSecTag2/HygroA-PD1-CTLA-4-treme) is then amplified, extracted, and purified.

### Antibody Expression and Purification-Diabodies

The pSecTag2/HygroA-PD1-CTLA-4-ipi or the pSecTag2/HygroA-PD1-CTLA-4-treme expression plasmid is transiently transfected into human kidney cell line 293T (ATCC Number: CRL-11268) with LipofectAMINE-plus (Invitrogen, Life Technologies) and cultured. The supernatant is sterilized with 0.22 μm PVDF filter, and concentrated using 40% PEG20000 solution. The concentrated supernatant is purified by HiTrap Chelating HP column (GE Healthcare, Piscataway, N.J.).

### DNA Constructs-Tribodies

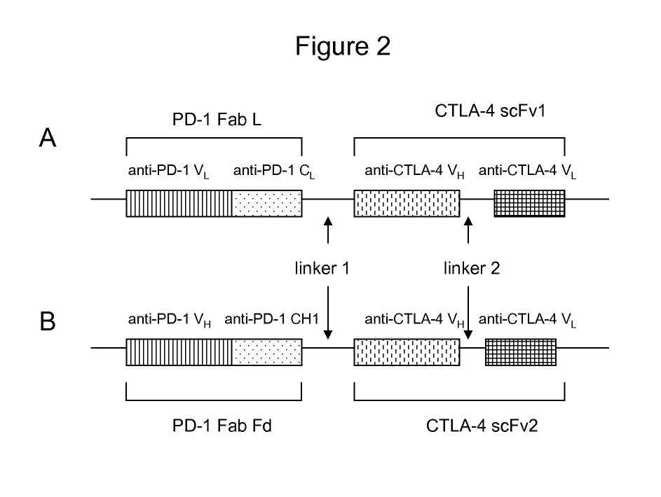

A pair of plasmids are required for the production of bispecific tribodies, as shown in FIG. 2.

Specifically, in the first plasmid shown in FIG. 2A, the variable light chain of an anti-human PD-1 antibody is fused to the constant region of a human kappa light chain (the fusion as shown in SEQ ID NO: 31), which is linked via a fifteen amino acid linker (linker 1, SEQ ID NO:33) to a variable heavy chain of an anti-CTLA-4 antibody (ipilimumab V_{H}, SEQ NO:5) which, in turn, is linked via another fifteen amino acid linker (linker 2, SEQ ID NO:34) to the variable light chain of anti-CTLA-4 antibody (ipilimumab V_{L}, SEQ NO:6). For ease of purification, a his-tag ((His)₆, SEQ ID NO:35) is added to the C-terminus of this construct (not shown).

In the second plasmid shown in FIG. 2B, the variable heavy chain of an anti-human PD-1 antibody is fused to the constant region 1 of a human IgG4 (the fusion as shown in SEQ ID NO:30), which is linked via a fifteen amino acid linker (linker 1, SEQ ID NO:33) to a variable heavy chain of an anti-CTLA-4 antibody (ipilimumab V_{H}, SEQ NO:5) which, in turn, is linked via another fifteen amino acid linker (linker 2, SEQ ID NO:34) to the variable light chain of anti-CTLA-4 antibody (ipilimumab V_{L}, SEQ NO:6). For ease of purification, a his-tag ((His)₆, SEQ ID NO:35) is also added to the C-terminus of this construct (not shown).

The two DNA segments encoding the bispecific tribodies are cloned into two separate expression vectors, pCAGGS (SEQ ID NO: 32) (De Sutter et al., 1992). The resulting plasmid pair encoding the bispecific tribody, pCAGGS-FabL-scFv-His₆ and pCAGGS-FabFd-scFv-His₆, are then amplified, extracted, and purified.

### Antibody Expression and Purification-Tribodies

For transient expression, HEK293T cells are transfected according to the Ca₃(PO₄)₂ precipitation method (O'Mahoney et al., 1994). Twenty hours prior to transfection, HEK293T cells are seeded at 4×10⁶ cells per 175 cm². Fourteen micrograms of DNA of each expression plasmid are added to the cells for 24 hours; the cells are covered with supplemented DMEM containing 5 mg/l bovine insulin, 5 mg/l transferrin and 5 μg/l selenium (ITS) replacing the FCS. Medium is harvested every 48 hours after transfection. For stable expression lines, SP2/0-Ag14 cells growing in log phase are harvested and resuspended at 4×10⁶ cells in 400 μl medium and kept on ice. Fifteen micrograms of linearized and purified plasmid is added to the cells in a 0.4 cm gap electroporation curvette and kept on ice for 1 min. A pulse (900 ρF, 250 V) is generated by an Easyject plus (Molecular Technologies, MO). Immediately, 1 ml of fresh medium is added and the cells are transferred to a 12 cm² culture plate. After 48 hour, the cells are incubated with medium containing both 0.6 mg/ml Zeocin® (Invitrogen, CA, USA) and 0.6 mg/ml G418 (Gibco BRL, UK) to select for plasmids containing either an L-chain and an Fd-chain derivative. After 30 days, the surviving cells are subcloned and the positive clones expanded.

The secreted Fab-scFv-(His)₆ protein is purified under native conditions from the culture supernatant using immobilized metal affinity chromatography (IMAC). The supernatant is filtered, 10 mM imidazol (pH 7.5) is added and it is subsequently applied to a 1 ml HiTrap chelating column (Amersham Pharmacia Biotech), loaded with Ni²⁺. After washing with 10 column volumes (CV) PBS, 50 mM imidazol, 10% glycerol (pH 7.5), the protein is eluted in 5 CV PBS, 400 mM imidazol, (pH 7.5). Finally, the protein is dialyzed to PBS. Gel filtration is performed on an XK 16/88 Superdex 200 column (Amersham Pharmacia Biotech, SE) calibrated with a commercial protein standard mix (BioRad, MA). A sample volume of 1 ml is loaded, and the column is developed in 15 mM NaH₂PO₄, 150 mM NaCl at 1 ml/min.

### Example 2

### In Vitro Assays

### Analysis of Antigen:Antibody Interactions

An amine coupling kit is obtained from GE Healthcare/Biacore (catalog number SR-I 000-50). The kit consists of 100 mM N-hydroxysuccinimide (NHS), 400 mM 1-ethyl-3-(3 dimethylaminopropyl) carbodiimide hydrochloride (EDC) and 1M ethanolamine hydrochloride-NaOH pH 8.5; EDC and NHS aliquots are stored at −20° C., ethanolamine at 0-4° C.; EDC and NHS are mixed 50:50 immediately prior to immobilization procedure.

Immobilization buffers of 10 mM sodium acetate (NaOAc) at pH 4.0, 4.5, 5.0 and 5.5 are used. The running buffer consists of 10 mM 4-(2-hydroxyethyl)-1-piperazineethanesulfonic acid (HEPES), 150 mM NaCl, 0.005% Tween 20, 3 mM ethylenediaminetetraacetic acid (EDTA), pH 7.2; filtered (0.2 μm) and de-gassed, 25° C.

Accessories include GE Healthcare/Biacore supplied plastic vials 7 mm (BR-1002-12), glass vials 9 mm (BR-1002-07), glass vials 16 mm (BR-1002-09), rubber caps type 2 (BR-1004-11), rubber caps type 3 (BR-1005-02), and CM5 sensorchips (BR-1003-99).

Biacore 2000 and BiaEvaluation software (v3.1) are used for data generation, processing, and analysis.

All testing and prep work are performed at room temperature (25° C.). The CM5 sensorchip surface is prepared using standard Biacore methodology. Briefly, after docking and priming with distilled water using the QUICKINJECT command, the CM5 surface is subjected to two consecutive 20 μl pulses each of 50 mM sodium hydroxide, 10 mM HCl, 0.1% sodium dodecyl sulfate (SDS) and 0.085% H₃PO₄ at a flow of 100 μl/min. Following the injections, there is a wash of the IFC and then priming with running buffer.

The amine coupling procedure/immobilization is performed according to Biacore standard methodology using a 5 μl/min flow rate. Briefly, to activate the CM5 surface, EDC and NHS are mixed 50:50, and the mixture is injected for 6 minutes (30 μl). Next the protein (CTLA-4, PD-1, or both CTLA-4, PD-1) is diluted in 10 mM NaOAc and injected over the desired flow cell. A 6 minute (30 μl) injection of ethanolamine follows.

The basic procedure for testing antibody binding to immobilized protein is performed using the KINJECT command to inject 20 μl of the bispecific antibody and follow dissociation for 120 sec. The flow rate used is 10 μl/min. Once the KINJECT command is completed, the surfaces are regenerated with either 0.02% SDS or a cocktail of EDTA, H₃PO₄, formic acid, MgCl₂ and guanidine HCl.

It is expected that the bispecific antibodies (the diabodies and the tribody) bind specifically to each of CTLA-4 and PD-1 proteins/antigens. The dissociation constant for binding of the bispecific antibody to each antigen will also be determined.

It is noted that the experiments set forth above may be employed to demonstrate binding of the bispecific antibodies to individual CTLA-4 and PD-1 antigens, it may not be able to show enhanced binding of the bispecific antibodies vs. monospecific antibodies (CTLA-4 antibody and PD-1 antibody individually) to multi-antigen presenting surfaces (surfaces containing both CTLA-4 and PD-1 proteins). It is noted, that initial testing with a model system employing GST-tagged and His-tagged proteins and corresponding biotin-derivatized anti-GST and anti-His antibodies failed to show enhanced binding. Very briefly, in the presence of 10 mM HEPES, 150 mM NaCl, 0.005% Tween 20, 3 mM EDTA, pH 7.2, the binding affinity/avidity of biotinylated anti-GST antibody to the GST-tagged protein ranged from 0.8-1.4 nM. Under these same conditions, for the biotinylated anti-His antibody binding to immobilized His-tagged protein, the range was 0.10-0.16 nM. The high affinities measured with each antigen-antibody pair were primarily a result of very slow off-rates and did not support demonstration of enhanced binding by linked antibodies. Numerous efforts to alter binding conditions to yield faster off-rates were unsuccessful. These data confirm that the a priori success or efficacy of a bispecific antibody cannot be predicted based on monotherapy or even concurrent monotherapy with two different antibody molecules. Nonetheless the inventors expect the methods and reagents disclosed herein to provide a superior and effective bispecific antibody.

### Binding of Bispecific Antibodies to Human T Cells

Peripheral blood mononuclear cells (PBMCs) are prepared by Histopaque density centrifugation from enriched lymphocyte preparations, which are obtainable from local blood banks or from fresh blood from healthy human donors. Human PBMCs are examined for PD-1 and CTLA-4 expression on various cell subsets by FACS. Biotinylated bispecific antibody is used in the assay. Bound antibody is detected using an PE-conjugated streptavidin. Flow cytometric analyses are performed using a FACScan flow cytometry (Becton Dickinson) and Flowjo software (Tree Star). PD-1 expression and CTLA-4 expression are expected to be detected on some peripheral human T cells, such as effector T cells.

### Effect of the Bispecific Antibodies on Function of T Regulatory Cells

T regulatory cells are lymphocytes that suppress the immune response. In this example, T regulatory cells are tested for its inhibitory function on proliferation and IFN-γ secretion of CD4⁺CD25⁺ T cells in the presence or absence of bispecific antibodies.

T regulatory cells are purified from PBMC using a CD4⁺CD25⁺ regulatory T cell isolation kit (Miltenyi Biotec Inc., Auburn, Calif.). T regulatory cells are added into a mixed lymphocyte reaction containing purified CD4⁺CD25⁺ T cells and allogeneic dendritic cells in a 2:1 ratio. Each bispecific antibody is added at a concentration of 10 μg/ml. Either no antibody or an isotype control antibody is used as a negative control. Culture supernatants are harvested on Day 5 for cytokine measurement using a Beadlyte cytokine detection system (Upstate Cell Signaling Solutions, Lake Placid, N.Y.). The cells are labeled with ³H-thymidine, cultured for another 18 hours, and analyzed for cell proliferation. It is expected that the addition of each bispecific antibody releases inhibition imposed by Treg cells on proliferation and IFN-γ secretion of CD4⁺CD25⁺ T cells, indicating that the bispecific antibodies have an effect on T regulatory cells.

### Effect of the Bispecific Antibodies on T Cell Activation

In this example, effect of blockade of CTLA-4 and PD-1 pathways by the bispecific antibody on T cell activation is examined. Purified human CD4+ T cells (Dynal CD4 T cell purification kit) are activated with 1 μg/ml soluble anti-CD3 antibody (BD) in the presence of autologous monocytes or monocyte-derived dendritic cells (DCs). Monocytes are purified using a Miltenyi CD14 monocyte purification kit, and DCs are generated in vitro after culture of monocytes with GM-CSF and IL-4 (PeproTech) for 7 days. After three days of activation in the presence or absence of a titrated bispecific antibody according to the present invention or irrelevant isotype control antibody, culture supernatants are harvested for ELISA analysis of IFNγ secretion while tritiated thymidine is added during the final 18 hours of the assay in order to measure T cell proliferation. It is expected that the simultaneous blockade of CTLA-4 and PD-1 pathways by each bispecific antibody of the present invention will result in enhanced T cell proliferation.

### Example 3

### In Vivo Efficacy Assays

Mice implanted with various tumor cell lines are treated in vivo with (i) vehicle, (ii) ipilimumab (iii) tremelimumab, (iv) an anti-PD1 antibody (whose V_{H} and V_{L} are listed as SEQ ID NOs: 9 and 10, respectively), (v) a combination of anti-PD-1 antibody and ipilimumab, (vi) a combination of anti-PD-1 antibody and tremelimumab, (vii) bispecific ipilimumab-PD-1 diabody, (viii) bispecific tremelimumab-PD-1 diabody, and (ix) bispecific ipilimumab-PD-1 tribody to examine the in vivo effect of these antibodies on (a) tumor establishment and growth and (b) the growth of established tumors.

### In Vivo Efficacy of Bispecific CTLA-4-PD1 Antibody on Mammary Carcinoma Establishment and Growth

The 4T1 mammary carcinoma is a transplantable tumor cell line originally isolated by Fred Miller and colleagues (Dexter et al., 1978; Aslakson and Miller, 1992). These experiments using the 4T1 cells are carried out using a modified protocol as disclosed in Pulaski et al., 2001. Briefly, 4T1 tumor cells are cultured in Iscove's Modified Dulbecco's Media (IMDM, Invitrogen, Carlsbad, Calif.), supplemented with 10% FBS and 1× antibiotic-antimycotic in a 37° C., 5% CO₂ tissue culture incubator. 8-week-old female BALB/c mouse (Harlan Laboratories) are injected subcutaneously (s.c.) in the mammary gland with 1×10⁶ 4 T1 cells on day 0. The mice are treated with PBS vehicle and the various antibodies listed above. The single antibody treatments are dosed at 10 mg/kg, the combination treatments of anti-CTLA-4 antibody and anti-PD-1 antibody are dosed at 5 mg/kg of each antibody (i.e., 10 mg/kg of total antibody), and the bispecific antibody treatments are dosed at 10 mg/kg. Antibody injections are then further administered on days 3, 6 and 10. The animals are euthanized when the tumor diameter reaches 14 to 16 mm or when the mice become moribund, according to IACUC guidelines.

It is expected that treatment with the bispecific antibody of the present invention has an in vivo inhibitory effect on mammary carcinoma establishment and growth that is greater than either antibody alone or a combination of anti-PD-1 antibody and anti-CTLA-4 antibody.

### In Vivo Efficacy of Bispecific CTLA-4-PD1 Antibody on Fibrosarcoma Establishment and Growth

For these tumor studies, female AJ mice between 6-8 weeks of age (Harlan Laboratories) are randomized by weight into 6 groups. The mice are implanted subcutaneously in the right flank with 2×10⁶ human fibrosarcoma cells (HT1080) dissolved in 200 μl of DMEM media on day 0. The mice are treated with PBS vehicle and the various antibodies listed above. The single antibody treatments are dosed at 10 mg/kg, the combination treatments of anti-CTLA-4 antibody and anti-PD-1 antibody are dosed at 5 mg/kg of each antibody (i.e., 10 mg/kg of total antibody), and the bispecific antibody treatments are dosed at 10 mg/kg. Antibody injections are then further administered on days 3, 6 and 10. The mice are monitored for tumor growth for approximately 6 weeks. Using an electronic caliper, the tumors are measured three dimensionally (height×width×length) and tumor volume is calculated. Mice will be euthanized when the tumors reach a tumor end point (1500 mm³) or show greater than 15% weight loss.

It is expected that treatment with the bispecific antibody of the present invention has an in vivo inhibitory effect on fibrosarcoma establishment and growth that is greater than either antibody alone or a combination of anti-PD-1 antibody and anti-CTLA-4 antibody.

### In Vivo Efficacy of Bispecific CTLA-4-PD1 Antibody on Colorectal Establishment and Growth

MDST8 colorectal cancer cells are implanted in C57BL/6 mice (2×10⁶ cells/mouse). On day 0 (i.e., the day the MDST8 cells are implanted in the mice), each group of mice is injected intraperitoneally (IP) with PBS vehicle and the various antibodies listed above. The single antibody treatments are dosed at 10 mg/kg, the combination treatments of anti-CTLA-4 antibody and anti-PD-1 antibody are dosed at 5 mg/kg of each antibody (i.e., 10 mg/kg of total antibody), and the bispecific antibody treatments are dosed at 10 mg/kg. Antibody injections are then further administered on days 3, 6 and 10. Using an electronic caliper, the tumors are measured three dimensionally (height×width×length) and tumor volume is calculated. Mice will be euthanized when the tumors reach a designated tumor end-point.

It is expected that treatment with the bispecific antibody according to the present invention will have an in vivo inhibitory effect on colorectal cancer cell establishment and growth that is greater than either antibody alone or a combination of anti-PD-1 antibody and anti-CTLA-4 antibody.

### In Vivo Efficacy of Bispecific CTLA-4-PD1 Antibody on Renal Cancer Establishment and Growth

Caki-1 renal cancer cells are implanted in C57BL/6 mice (2×10⁶ cells/mouse). On day 0 (i.e., the day the Caki-1 cells are implanted in the mice), each groups of mice is injected intraperitoneally (IP) with PBS vehicle and the various antibodies listed above. The single antibody treatments are dosed at 10 mg/kg, the combination treatments of anti-CTLA-4 antibody and anti-PD-1 antibody are dosed at 5 mg/kg of each antibody (i.e., 10 mg/kg of total antibody), and the bispecific antibody treatments are dosed at 10 mg/kg. Antibody injections are then further administered on days 3, 6 and 10. Using an electronic caliper, the tumors are measured three dimensionally (height×width×length) and tumor volume is calculated. Mice will be euthanized when the tumors reach a designated tumor end-point.

It is expected that treatment with the bispecific antibody according to the present invention will have an in vivo inhibitory effect on renal cancer cell establishment and growth that is greater than either antibody alone or a combination of anti-PD-1 antibody and anti-CTLA-4 antibody.

### In Vivo Efficacy of Bispecific CTLA-4-PD1 Antibody on Lung Cancer Establishment and Growth

WX322 lung cancer cells are implanted in C57BL/6 mice (2×10⁶ cells/mouse). On day 0 (i.e., the day the lung cells are implanted in the mice), each groups of mice is injected intraperitoneally (IP) with PBS vehicle and the various antibodies listed above. The single antibody treatments are dosed at 10 mg/kg, the combination treatments of anti-CTLA-4 antibody and anti-PD-1 antibody are dosed at 5 mg/kg of each antibody (i.e., 10 mg/kg of total antibody), and the bispecific antibody treatments are dosed at 10 mg/kg. Antibody injections are then further administered on days 3, 6 and 10. Using an electronic caliper, the tumors are measured three dimensionally (height×width×length) and tumor volume is calculated. Mice will be euthanized when the tumors reach a designated tumor end-point.

It is expected that treatment with the bispecific antibody according to the present invention will have an in vivo inhibitory effect on lung cancer cell establishment and growth that is greater than either antibody alone or a combination of anti-PD-1 antibody and anti-CTLA-4 antibody.

### In Vivo Efficacy of Bispecific CTLA-4-PD1 Antibody on the Growth of Established Melanoma

Here, the in vivo effect of various antibodies on established tumor growth is examined.

HRLN female nu/nu mice are injected with 1×10⁷ A2058 tumor cells (melanoma) in 50% Matrigel subcutaneously into the flank. The injection volume is 0.2 mL/mouse. Age of the mice at the start of the experiment is 8 to 12 weeks. Body weight is measured biweekly, starting on day 4, until the end of the experiment. Tumor size is also measured biweekly, starting on day 4, until the end of the experiment. Animals are monitored individually. The endpoint of the experiment is a tumor volume of 2000 mm³ or 17 days, whichever comes first. Responders can be followed longer. When the endpoint is reached, the animals are euthanized.

Xenograft measures are typically aggregated in a ‘carry-forward’ analysis: for subjects missing at a given time point due to sacrifice, the largest tumor measurement from the nearest earlier assessment will be used to represent the subject at that later day. With group estimates across the all xenograft lines, a standard one-way ANOVA analysis, with a post-hoc Dunnett multiple testing comparison, is used to identify lines which show growth difference. Significance is assessed at p values less than 0.05.

On days 4, 7, 10, 14, and 17 post-implantation, each group of mice is injected intraperitoneally (IP) with PBS vehicle and the various antibodies listed above. The single antibody treatments are dosed at 10 mg/kg, the combination treatments of anti-CTLA-4 antibody and anti-PD-1 antibody are dosed at 5 mg/kg of each antibody (i.e., 10 mg/kg of total antibody), and the bispecific antibody treatments are dosed at 10 mg/kg. Antibody injections are then further administered on days 3, 6 and 10.

It is expected that treatment with the bispecific antibody according to the present invention will have an in vivo inhibitory effect on the growth of established melanoma that is greater than either antibody alone or a combination of anti-PD-1 antibody and anti-CTLA-4 antibody.

### In Vivo Efficacy of Bispecific CTLA-4-PD1 Antibody on the Growth of Established Fibrosarcoma

Human fibrosarcoma cells (HT1080) are implanted subcutaneously in NJ mice (2×10⁶ cells/mouse) on day 0. On day 6, the tumors are formed. On days 7, 10, 14, and 17 post-implantation, mice are injected IP with vehicle and various antibodies as set forth above. The single antibody treatments are dosed at 10 mg/kg, the combination treatments of anti-CTLA-4 antibody and anti-PD-1 antibody are dosed at 5 mg/kg of each antibody (i.e., 10 mg/kg of total antibody), and the bispecific antibody treatments are dosed at 10 mg/kg. The study is expected to last about 50 days, and tumor measurements are taken on various days throughout the course of the study. Tumor volume is calculated by measuring tumors in three dimensions (height×width×length) using an electronic caliper. Mice will be euthanized when the tumors reach a designated tumor end-point—a volume of 1500 mm³ and/or an ulcerated tumor.

It is expected that treatment with the bispecific antibody according to the present invention will have an in vivo inhibitory effect on the growth of established fibrosarcoma that is greater than either antibody alone or a combination of anti-PD-1 antibody and anti-CTLA-4 antibody.

### In Vivo Efficacy of Bispecific CTLA-4-PD1 Antibody on the Growth of Established Colorectal Cancer Cells

MDST8 colorectal cancer cells are implanted in C57BL/6 mice (2×10⁶ cells/mouse) on day 0. On day 6, the tumors are formed. On days 7, 10, 14, and 17 post-implantation, mice are injected IP with vehicle and various antibodies as set forth above. The single antibody treatments are dosed at 10 mg/kg, the combination treatments of anti-CTLA-4 antibody and anti-PD-1 antibody are dosed at 5 mg/kg of each antibody (i.e., 10 mg/kg of total antibody), and the bispecific antibody treatments are dosed at 10 mg/kg. Using an electronic caliper, the tumors are measured three dimensionally (height×width×length), and tumor volume is calculated. Tumor measurements are taken at the beginning of treatment (i.e., on day 7) and on days 10, 13, 17, 20, 24 and 27 post-antibody treatment. Mice will be euthanized when the tumors reach a designated tumor end-point (a particular tumor volume such as 1500 mm³ and/or when the mice show greater than about 15% weight loss).

It is expected that treatment with the bispecific antibody according to the present invention will have an in vivo inhibitory effect on the growth of established fibrosarcoma that is greater than either antibody alone or a combination of anti-PD-1 antibody and anti-CTLA-4 antibody.

Tumor Immunity in Mice Following Bispecific Antibody Treatment and Re-Challenge with Tumor Cells

Mice that survive tumor-free from a challenge with tumor cells and treatment with the bispecific antibody (i.e., treatment similar to the efficacy studies set forth above) will then be re-challenged with tumor cells to investigate immunity to tumor formation after such a treatment.

The previously treated, tumor-free mice are re-challenged by subcutaneously implanting 1×10⁶ of tumor cells from the same cell line as the first challenge. As a control, naive mice are subcutaneously implanted with 1×10⁶ of tumor cells per mouse. Tumor formation and volume are monitored with a precision electronic caliper twice a week until three months post second implantation. It is expected that the tumor-free mice re-challenged with tumor cells will not develop tumors during this period of time. It is expected that this data will indicate that the bispecific antibody therapy according to the present invention will produce a persistent immunity to tumor relapse.

### Example 4

### In Vivo Dose Titration Assays

### Dose Titration of Bispecific Antibody on Established Tumor Growth

Mice implanted with human tumor cells (or xenograft lines) are treated in vivo with (i) vehicle, (ii) 0.5, 1, 5, 10, 30, and 50 mg/kg of an anti-PD-1 antibody (whose V_{H} and V_{L} are listed as SEQ ID NOs: 9 and 10, respectively), (iii) 0.5, 1, 5, 10, 30, and 50 mg/kg of ipilimumab, (iv) 0.25, 0.5, 2.5, 5, 15, and 25 mg/kg of each of the anti-PD-1 antibody and an ipilimumab (or 0.5, 1, 5, 10, 30, and 50 mg/kg of the combined antibodies), (v) 0.5, 1, 5, 10, 30, and 50 mg/kg of the bispecific ipilimumab-PD-1 diabody, and (vi) 0.5, 1, 5, 10, 30, and 50 mg/kg of the bispecific tribody to examine the in vivo effect of these antibodies on (a) tumor establishment and growth and (b) the growth of established tumors. The protocols for such treatments are as set forth above in Example 3.

It is expected that treatment with the bispecific antibodies of the present invention will have an in vivo inhibitory effect on the growth of established tumors that is greater than either antibody alone or a combination of anti-PD-1 antibody and anti-CTLA-4 antibody at a comparable dose.

### Example 5

### Therapy with Bispecific Antibodies in Human Patients

Eligible patients are at least 18 years of age; have received a diagnosis of measurable, unrespectable, stage III or IV melanoma; have an Eastern Cooperative Oncology Group performance status of 0 (asymptomatic) or 1 (ambulatory but restricted in strenuous activity); have adequate organ function; and have a life expectancy of at least 4 months. Exclusion criteria are active, untreated central nervous system metastasis, a history of autoimmune disease, previous therapy with T-cell modulating antibodies (excluding ipilimumab for patients in the sequenced-regimen cohorts), human immunodeficiency virus infection, and hepatitis B or C infection.

In the diabody-regimen cohorts, patients are treated with escalating doses of intravenous bispecific ipilimumab-PD-1 diabody every 6 weeks for eight doses. The treatment is subsequently continued every 12 weeks for up to eight doses. In this regimen group, cohort 1 is designated to receive 0.6 mg of bispecific antibody per kilogram of body weight; cohort 2, 1 mg of bispecific antibody per kilogram; cohort 3, 2 mg of bispecific antibody per kilogram; cohort 4, 6 mg of bispecific antibody per kilogram; cohort 5, 10 mg of bispecific antibody per kilogram.

In the tribody-regimen cohorts, patients are treated with escalating doses of intravenous bispecific tribody every 6 weeks for eight doses. The treatment is subsequently continued every 12 weeks for up to eight doses. In this regimen group, cohort 1 is designated to receive 0.6 mg of tribody per kilogram of body weight; cohort 2, 1 mg of bispecific antibody per kilogram; cohort 3, 2 mg of bispecific antibody per kilogram; cohort 4, 6 mg of bispecific antibody per kilogram; cohort 5, 10 mg of bispecific antibody per kilogram.

In the combined treatment-regimen cohorts, patients are treated with escalating doses of intravenous PD-1 and CTLA-4 antibodies every 6 weeks for eight doses. The treatment is subsequently continued every 12 weeks for up to eight doses. Within a cohort, doses of PD-1 and CTLA-4 antibodies are kept constant. When the two drugs are administered together, anti-PD-1 antibody is administered first. In this regimen group, cohort 1 is designated to receive 0.3 mg of PD-1 antibody per kilogram of body weight and 0.3 mg of CTLA-4 antibody per kilogram; cohort 2, 1 mg of PD-1 antibody per kilogram and 1 mg of CTLA-4 antibody per kilogram; cohort 3, 3 mg of PD-1 antibody per kilogram and 3 mg of CTLA-4 antibody per kilogram.

Patients may be followed for a total of 2.5 years after the initiation of therapy. Patients with a complete response, a partial response, or stable disease for at least 24 weeks and subsequent disease progression may be retreated with the original regimen. Disease assessment is performed per protocol, with the use of computed tomography or magnetic resonance imaging, as appropriate. For both regimen groups, tumor responses are adjudicated with the use of modified World Health Organization (WHO) criteria and immunerelated criteria. Tumor assessments are performed at week 8 and then every 8 weeks thereafter. The safety evaluation is performed per protocol. The severity of adverse events is graded according to the National Cancer Institute Common Terminology Criteria for Adverse Events, version 3.0.

The period for evaluating dose-limiting toxicity for the purposes of dose escalation is 9 weeks. No dose escalation is allowed in an individual patient, and patients who had dose-limiting adverse events are to discontinue therapy.

Adverse events are coded with the use of the Medical Dictionary for Regulatory Activities (MedDRA), version 15.1. Selected adverse events with potential immunologic causes and those that require more frequent monitoring or intervention with immune suppression or hormone replacement are identified with the use of a predefined list of MedDRA terms. These are similar to events previously described as immune-related adverse events or adverse events of special interest. Best overall responses are derived programmatically from tumor measurements provided by the study-site radiologist and investigators according to the modified WHO criteria or immune-related response criteria. Complete and partial responses are confirmed by means of at least one subsequent tumor assessment. The magnitude of the reduction in target lesions is assessed radiographically. A response is characterized as “deep” if a reduction of 80% or more from the baseline measurements is noted.

It is expected that the bispecific antibodies (both the diabody and the tribody) according to the present invention will be better tolerated and more efficacious than the combined treatment using PD-1 and CTLA-4 antibodies.

### DOCUMENTS

ASLAKSON, C. J. and Miller, F. R. 1992. Selective events in the metastatic process defined by analysis of the sequential dissemination of subpopulations of a mouse mammary tumor. Cancer Res. 52:1399-1405.
CURRAN M A, Kim M, Montalvo W, Al-Shamkhani A, Allison J P (2011) Combination CTLA-4 Blockade and 4-1 BB Activation Enhances Tumor Rejection by Increasing T-Cell Infiltration, Proliferation, and Cytokine Production. PLoS ONE 6(4): e19499.
CURRAN, Michael A., et al. “PD-1 and CTLA-4 combination blockade expands infiltrating T cells and reduces regulatory T and myeloid cells within B16 melanoma tumors.” Proceedings of the National Academy of Sciences 107.9 (2010): 4275-4280.
DURAISWAMY et al. “Dual blockade of PD-1 and CTLA-4 combined with Tumor Vaccine Effectively Restores T-Cell Rejection Function in Tumors.” Cancer Res. (Published OnlineFirst Apr. 30, 2013)
DARIAVACH, P., Mattéi, M.-G., Golstein, P. and Lefranc, M.-P. (1988), Human Ig superfamily CTLA-4 gene: chromosomal localization and identity of protein sequence between murine and human CTLA-4 cytoplasmic domains. Eur. J. Immunol., 18: 1901-1905.
DE SUTTER, Kristine, et al. “Production of functionally active murine and murine:: human chimeric F(ab′)₂ fragments in COS-1 cells.” Gene 113.2 (1992): 223-230.
DEXTER, D. L., Kowalski, H. M., Blazar, B. A., Fligiel, Z., Vogel, R., and Heppner, G. H. 1978. Heterogeneity of tumor cells from a single mouse mammary tumor. Cancer Res. 38:3174-3181.
HUDSON, P. J., Alexander A Kortt, High avidity scFv multimers; diabodies and triabodies, Journal of Immunological Methods, Volume 231, Issues 1-2, 10 Dec. 1999, Pages 177-189.
O'MAHONEY, John et al., “Optimization of Experimental Variables Influencing Reporter Gene Expression in Hepatoma Cells Following Calcium Phosphate Transfection” DNA and Cell Biology 13.12 (1994): 1227-1232.
Mekhaiel, David N A, et al. “Polymeric human Fc-fusion proteins with modified effector functions.” Scientific reports 1 (2011).
PULASKI, B. A. and Ostrand-Rosenberg, S. 2001. Mouse 4T1 Breast Tumor Model. Current Protocols in Immunology. 39:20.2.1-20.2.16.
SHINOHARA, T., Masafumi Taniwaki, Yasumasa Ishida, Masashi Kawaichi, Tasuku Honjo, Structure and Chromosomal Localization of the Human PD-1 Gene (PDCD1), Genomics, Volume 23, Issue 3, October 1994, Pages 704-706, ISSN 0888-7543
WOLCHOK et al., Nivolumab plus Ipilimumab in Advanced Melanoma. New England J. of Med. Jun. 2, 2013.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A bispecific antibody comprising:
(a) a first antigen binding moiety that specifically binds an epitope on human cytotoxic T-lymphocyte-associated antigen 4 (CTLA-4), the first antigen binding moiety comprising an antibody having:
(1) a heavy chain CDR1 comprising SYTMH (SEQ ID NO:21), a heavy chain CDR2 comprising FISYDGNNKYYADSVKG (SEQ ID NO:22), and a heavy chain CDR3 comprising TGWLGPFDY (SEQ ID NO:23); and
(2) a light chain CDR1 comprising RASQSVGSSYLA (SEQ ID NO:18), a light chain CDR2 comprising GAFSRAT (SEQ ID NO:19), and a light chain CDR3 comprising QQYGSSPWT (SEQ ID NO:20); and
(b) a second antigen binding moiety that specifically binds an epitope on a human programmed death 1 (PD-1) receptor, the second antigen binding moiety comprising an antibody having:
(1) a heavy chain CDR1 comprising NSGMH (SEQ ID NO:27), a heavy chain CDR2 comprising VIWYDGSKRYYADSVKG (SEQ ID NO:28), and a heavy chain CDR3 comprising NDDYW (SEQ ID NO:29); and
(2) a light chain CDR1 comprising RASQSVSSYL (SEQ ID NO:24), a light chain CDR2 comprising DASNRAT (SEQ ID NO:25), and a light chain CDR3 comprising QQSSNWPRT (SEQ ID NO:26).

2. The bispecific antibody of claim 1, wherein the first antigen binding moiety specifically binds an epitope in the extracellular IgV domain of the human CTLA-4.

3. The bispecific antibody of claim 1, wherein the second antigen binding moiety specifically binds an epitope in the extracellular IgV domain of the human PD-1 receptor.

4. The bispecific antibody of claim 1, which is a recombinant antibody, a monoclonal antibody, a chimeric antibody, a humanized antibody, a human antibody, or an antibody fragment.

5. The bispecific antibody of claim 1 in which the first antigen binding moiety comprises a variable heavy chain as depicted in SEQ ID NO:5, a variable light chain as depicted in SEQ ID NO:6 and the second antigen binding moiety comprises a variable heavy chain as depicted in SEQ ID NO:11, a variable light chain as depicted in SEQ ID NO:12.

6. The bispecific antibody of claim 1, wherein the first and second first antigen binding moieties are connected directly or by a linker.

7. The bispecific antibody of claim 6, wherein the linker is selected from the group consisting of a chemical linker or a polypeptide linker.

8. The bispecific antibody of claim 1, wherein the bispecific antibody is bivalent, trivalent, or tetravalent.

9. A bispecific antibody comprising:
(a) a first antigen binding moiety that specifically binds an epitope on human cytotoxic T-lymphocyte-associated antigen 4 (CTLA-4), and
(b) a second antigen binding moiety that specifically binds an epitope on a human programmed death 1 (PD-1) receptor.

10. The bispecific antibody of claim 9, wherein the first antigen binding moiety specifically binds an epitope in the extracellular IgV domain of the human CTLA-4.

11. The bispecific antibody of claim 9, wherein the second antigen binding moiety specifically binds an epitope in the extracellular IgV domain of the human PD-1 receptor.

12. The bispecific antibody of claim 9, which is a recombinant antibody, a monoclonal antibody, a chimeric antibody, a humanized antibody, a human antibody, or an antibody fragment.

13. The bispecific antibody of claim 9, wherein the first and second antigen binding moieties are connected directly or by a linker.

14. The bispecific antibody of claim 13, wherein the linker is selected from the group consisting of a chemical linker or a polypeptide linker.

15. The bispecific antibody of claim 9, wherein the first antigen binding moiety comprises a variable heavy chain and a variable light chain of ipilimumab, and the second antigen binding moiety comprises a variable heavy chain and a variable light chain of nivolumab.

16. The bispecific antibody of claim 9, wherein the first antigen binding moiety comprises a variable heavy chain and a variable light chain of tremelimumab, and the second antigen binding moiety comprises a variable heavy chain and a variable light chain of nivolumab.

17. The bispecific antibody of claim 9, wherein each antigen binding moiety is independently selected from the group consisting of IgM, IgG, IgD, IgA, IgE, antibody fragments that retain antigen recognition and binding capability that are Fab, Fab′, F(ab′)₂, and Fv fragments, and combinations thereof, and further wherein the first and second antigen binding moieties are connected directly or by a linker.

18. The bispecific antibody of claim 9, wherein the bispecific antibody is bivalent, trivalent, or tetravalent.

19. The bispecific antibody of claim 9, wherein the bispecific antibody is selected from the group consisting of a tandem scFv (taFv or scFv₂), diabody, dAb₂/VHH₂, knob-into-holes derivates, SEED-IgG, heteroFc-scFv, Fab-scFv, scFv-Jun/Fos, Fab′-Jun/Fos, tribody, DNL-F(ab)₃, scFv₃-CH1/CL, Fab-scFv₂, IgG-scFab, IgG-scFv, scFv-IgG, scFv₂-Fc, F(ab′)₂-scFv₂, scDB-Fc, scDb-CH3, Db-Fc, scFv₂-H/L, DVD-Ig, tandAb, scFv-dhlx-scFv, dAb₂-IgG, dAb-IgG, dAb-Fc-dAb, and combinations thereof.

20. The bispecific antibody of claim 9, wherein the bispecific antibody is a diabody or a tribody.

21. A pharmaceutical composition comprising a bispecific antibody of claim 1 and a pharmaceutically acceptable excipient.

22. A pharmaceutical composition comprising a bispecific antibody of claim 5 and a pharmaceutically acceptable excipient.

23. A pharmaceutical composition comprising a bispecific antibody of claim 9 and a pharmaceutically acceptable excipient.

24. A method of treating cancer in a subject, comprising administering to the subject a therapeutically effective amount of the pharmaceutical composition of claim 21.

25. The method according to claim 24, wherein the cancer is selected from the group consisting of melanoma, lung cancer, and renal cancer.

26. The method according to claim 25, wherein the cancer is melanoma.

27. The method according to claim 25, wherein the subject is human.

28. A method of treating cancer in a subject comprising administering to the subject a therapeutically effective amount of the pharmaceutical composition of claim 22.

29. The method according to claim 28, wherein the cancer is selected from the group consisting of melanoma, lung cancer, and renal cancer.

30. The method according to claim 29, wherein the cancer is melanoma.

31. The method according to claim 29, wherein the subject is human.

32. A method of treating cancer in a subject comprising administering to the subject a therapeutically effective amount of the pharmaceutical composition of claim 23.

33. The method according to claim 32, wherein the cancer is selected from the group consisting of melanoma, lung cancer, and renal cancer.

34. The method according to claim 33, wherein the cancer is melanoma.

35. The method according to claim 33, wherein the subject is human.

36. A method of treating cancer in a subject comprising administering to the subject a therapeutically effective amount of a bispecific antibody one antigen binding moiety of which specifically binds human CTLA-4 and the other antigen binding moiety of which binds to human PD-1 receptor.

37. A method of treating melanoma in a subject comprising administering to the subject a therapeutically effective amount of at least one isolated bispecific antibody comprising a first antigen binding moiety that specifically binds an epitope in the extracellular IgV domain of the human CTLA-4 and a second antigen binding moiety that specifically binds an epitope in the extracellular IgV domain of the human PD-1 receptor.

38. The method according to claim 37, wherein the first antigen binding moiety comprises a heavy chain and a light chain of ipilimumab and the second antigen binding moiety comprises a heavy chain and a light chain of nivolumab.

39. The method according to claim 38 in which from about 0.3-10 mg/kg of the bispecific antibody is administered to the subject.

40. The method according to claim 39 in which about 0.3-2.5 mg/kg of the bispecific antibody is administered to the subject.

41. The method according to claim 39 in which less than about 1 mg/kg of the bispecific antibody is administered to the subject.

42. The method according to claim 38, further comprising administering to the subject a therapeutically effective amount of an ipilimumab.

43. The method according to claim 42, wherein about 0.3-1 mg/kg of the bispecific antibody and about 1-2 mg/kg of the ipilimumab is administered to the subject.

44. A kit for treating a cancer in a subject comprising the pharmaceutical composition according to claim 21.

45. A bispecific antibody comprising:
(a) a first antigen binding moiety that specifically binds an epitope on human cytotoxic T-lymphocyte-associated antigen 4 (CTLA-4), and
(b) a second antigen binding moiety that specifically binds an epitope on a human programmed death ligand 1 (PD-L1).

46. A pharmaceutical composition comprising the bispecific antibody according to claim 45 and a pharmaceutically acceptable excipient.

47. A kit for treating a cancer in a subject comprising the pharmaceutical composition according to claim 46.

48. A method of treating cancer in a subject comprising administering to the subject a therapeutically effective amount of a bispecific antibody one antigen binding moiety of which specifically binds human CTLA-4 and the other antigen binding moiety of which binds to human PD-L1.
